SEQUENCE LISTING (137 sequences)

SEQ ID NO: 1 - protein, 652 aa, Homo sapiens
MATSMGLLLLLLLLLTQPGAGTGADTEAVVCVGTACYTAHSGKLSAAEAQNHCNQNGGNLATVKSKEEAQHVQRVLAQLLRREAALTARMSKFWIGLQREKGKCLDPSLPLKGFSWVGGGEDTPYSNWHKELRNSCISKRCVSLLLDLSQPLLPSRLPKWSEGPCGSPGSPGSNIEGFVCKFSFKGMCRPLALGGPGQVTYTTPFQTTSSSLEAVPFASAANVACGEGDKDETQSHYFLCKEKAPDVFDWGSSGPLCVSPKYGCNFNNGGCHQDCFEGGDGSFLCGCRPGFRLLDDLVTCASRNPCSSSPCRGGATCVLGPHGKNYTCRCPQGYQLDSSQLDCVDVDECQDSPCAQECVNTPGGFRCECWVGYEPGGPGEGACQDVDECALGRSPCAQGCTNTDGSFHCSCEEGYVLAGEDGTQCQDVDECVGPGGPLCDSLCFNTQGSFHCGCLPGWVLAPNGVSCTMGPVSLGPPSGPPDEEDKGEKEGSTVPRAATASPTRGPEGTPKATPTTSRPSLSSDAPITSAPLKMLAPSGSPGVWREPSIHHATAASGPQEPAGGDSSVATQNNDGTDGQKLLLFYILGTVVAILLLLALALGLLVYRKRRAKREEKKEKKPQNAADSYSWVPERAESRAMENQYSPTPGTDC

SEQ ID NO: 2 - protein, 742 aa, Mus musculus
MKDDFAEEEEVQSFGYKRFGIQEGTQCTKCKNNWALKFSIVLLYILCALLTITVAILGYKVVEKMDNVTDGMETSHQTYDNKLTAVESDLKKLGDQAGKKALSTNSELSTFRSDILDLRQQLQEITEKTSKNKDTLEKLQANGDSLVDRQSQLKETLQNNSFLITTVNKTLQAYNGYVTNLQQDTSVLQGNLQSQMYSQSVVIMNLNNLNLTQVQQRNLISNLQQSVDDTSLAIQRIKNDFQNLQQVFLQAKKDTDWLKEKVQSLQTLAANNSALAKANNDTLEDMNSQLSSFTGQMDNITTISQANEQSLKDLQDLHKDTENRTAVKFSQLEERFQVFETDIVNIISNISYTAHHLRTLTSNLNDVRTTCTDTLTRHTDDLTSLNNTLVNIRLDSISLRMQQDMMRSKLDTEVANLSVVMEEMKLVDSKHGQLIKNFTILQGPPGPRGPKGDRGSQGPPGPTGNKGQKGEKGEPGPPGPAGERGTIGPVGPPGERGSKGSKGSQGPKGSRGSPGKPGPQGPSGDPGPPGPPGKDGLPGPQGPPGFQGLQGTVGEPGVPGPRGLPGLPGVPGMPGPKGPPGPPGPSGAMEPLALQNEPTPASEVNGCPPHWKNFTDKCYYFSLEKEIFEDAKLFCEDKSSHLVFINSREEQQWIKKHTVGRESHWIGLTDSEQESEWKWLDGSPVDYKNWKAGQPDNWGSGHGPGEDCAGLIYAGQWNDFQCDEINNFICEKEREAVPSSIL

SEQ ID NO: 3 - protein, 371 aa, Bos taurus
MLLLPLSVLLLLTQPWRSLGAEMKIYSQKTLANGCTLVVCRPPEGGLPGRDGQDGREGPQGEKGDPGSPGPAGRAGRPGPAGPIGPKGDNGSAGEPGPKGDTGPPGPPGMPGPAGREGPSGKQGSMGPPGTPGPKGDTGPKGGMGAPGMQGSPGPAGLKGERGAPGELGAPGSAGVAGPAGAIGPQGPSGARGPPGLKGDRGDPGERGAKGESGLADVNALKQRVTILEGQLQRLQNAFSRYKKAVLFPDGQAVGKKIFKTAGAVKSYSDAQQLCREAKGQLASPRSAAENEAVAQLVRAKNNDAFLSMNDISTEGKFTYPTGESLVYSNWASGEPNNNNAGQPENCVQIYREGKWNDVPCSEPLLVICEF

SEQ ID NO: 4 - protein, 371 aa, Bos taurus; identical to SEQ ID NO: 3

SEQ ID NO: 5 - protein, 420 aa, Mus musculus
MNGFRVLLRSNLSMLLLLALLHFQSLGLDVDSRSAAEVCATHTISPGPKGDDGERGDTGEEGKDGKVGRQGPKGVKGELGDMGAQGNIGKSGPIGKKGDKGEKGLLGIPGEKGKAGTICDCGRYRKVVGQLDISVARLKTSMKFIKNVIAGIRETEEKFYYIVQEEKNYRESLTHCRIRGGMLAMPKDEVVNTLIADYVAKSGFFRVFIGVNDLEREGQYVFTDNTPLQNYSNWKEEEPSDPSGHEDCVEMLSSGRWNDTECHLTMYFVSSLQEDLIEDCLREQGLLVQVTPANQELLFGIDTFLGPMSCVYQRTGTKQKLYSQCRLWDGLAKKQTNETANIATFCKGAEPNRGSRPCGQKQEMMTLMMSGNKGITTFPESDNLFKWVGTMLGAAGTIDEDLKYKLSLNSPVVTLIIHPQ

SEQ ID NO: 6 - protein, 742 aa, Mus musculus; identical to SEQ ID NO: 2

SEQ ID NO: 7 - protein, 251 aa, Danio rerio
MALLKLFLGALLLLQLVLQLMAGAADPQSLNCPAYAGVPGTPGHNGLPGRDGRVGRDGANGPKGEKGEPGVNVQGPPGKAGPPGPAGAKGERGPSGLPGQDCMSDSLKSELQKLSDKIALIEKVVNFKTFKKVGQKYYVTDDVEETFDKGMQYCSSNGGALVLPRTLEENALLKVFVSSAFKRLFIRITDREKEGEFVDTDRKKLTFTNWGPNQPDNYKGAQDCGAIADSGLWDDVSCDSLYPIICEIEIK

SEQ ID NO: 8 - protein, 742 aa, Homo sapiens
MKDDFAEEEEVQSFGYKRFGIQEGTQCTKCKNNWALKFSIILLYILCALLTITVAILGYKVVEKMDNVTGGMETSRQTYDDKLTAVESDLKKLGDQTGKKAISTNSELSTFRSDILDLRQQLREITEKTSKNKDTLEKLQASGDALVDRQSQLKETLENNSFLITTVNKTLQAYNGYVTNLQQDTSVLQGNLQNQMYSHNVVIMNLNNLNLTQVQQRNLITNLQRSVDDTSQAIQRIKNDFQNLQQVFLQAKKDTDWLKEKVQSLQTLAANNSALAKANNDTLEDMNSQLNSFTGQMENITTISQANEQNLKDLQDLHKDAENRTAIKFNQLEERFQLFETDIVNIISNISYTAHHLRTLTSNLNEVRTTCTDTLTKHTDDLTSLNNTLANIRLDSVSLRMQQDLMRSRLDTEVANLSVIMEEMKLVDSKHGQLIKNFTILQGPPGPRGPRGDRGSQGPPGPTGNKGQKGEKGEPGPPGPAGERGPIGPAGPPGERGGKGSKGSQGPKGSRGSPGKPGPQGPSGDPGPPGPPGKEGLPGPQGPPGFQGLQGTVGEPGVPGPRGLPGLPGVPGMPGPKGPPGPPGPSGAVVPLALQNEPTPAPEDNGCPPHWKNFTDKCYYFSVEKEIFEDAKLFCEDKSSHLVFINTREEQQWIKKQMVGRESHWIGLTDSERENEWKWLDGTSPDYKNWKAGQPDNWGHGHGPGEDCAGLIYAGQWNDFQCEDVNNFICEKDRETVLSSAL

SEQ ID NO: 9 - protein, 622 aa, Homo sapiens
MKDDFAEEEEVQSFGYKRFGIQEGTQCTKCKNNWALKFSIILLYILCALLTITVAILGYKVVEKMDNVTGGMETSRQTYDDKLTAVESDLKKLGDQTGKKAISTNSELSTFRSDILDLRQQLREITEKTSKNKDTLEKLQASGDALVDRQSQLKETLENNSFLITTVNKTLQAYNGYVTNLQQDTSVLQGNLQNQMYSHNVVIMNLNNLNLTQVQQRNLITNLQRSVDDTSQAIQRIKNDFQNLQQVFLQAKKDTDWLKEKVQSLQTLAANNSALAKANNDTLEDMNSQLNSFTGQMENITTISQANEQNLKDLQDLHKDAENRTAIKFNQLEERFQLFETDIVNIISNISYTAHHLRTLTSNLNEVRTTCTDTLTKHTDDLTSLNNTLANIRLDSVSLRMQQDLMRSRLDTEVANLSVIMEEMKLVDSKHGQLIKNFTILQGPPGPRGPRGDRGSQGPPGPTGNKGQKGEKGEPGPPGPAGERGPIGPAGPPGERGGKGSKGSQGPKGSRGSPGKPGPQGPSGDPGPPGPPGKEGLPGPQGPPGFQGLQGTVGEPGVPGPRGLPGLPGVPGMPGPKGPPGPPGPSGAVVPLALQNEPTPAPEDNSKSKPSLQPGGQGSACA

SEQ ID NO: 10 - protein, 742 aa, Mus musculus
MKDDFAEEEEVQSFGYKRFGIHEGTQCTKCINNWALKFSIVLLYILCALLTITVAILGYKVVEKMDNVSDGMETSHQTYDNKLTAVESDLKKLGDQAGKKALSTNSELSTFRSDILDLRQQLQEITEKTSKNKDTLEKLQANGDSLVDRQSQLKETLQNNSFLITTVNKTLQAYNGYVTNLQQDTNVLQGNLQSQMYSQSVVIMNLNNLNLTQVQQRNLISNLQQSVDDTSLAIQRIKNDFQNLQQVFLQAKKDTDWLKEKVQSLQTLAANNSALAKANNDTLEDMNSQLSSFTGQMDNITTISQANEQSLKDLQDLHKDTENRTAVKFSQLEERFQVFETDIVNIISNISYTAHHLRTLTSNLNDVWTTCTDTLTRHTDDLTSLNNTLVNIRLDSISLRMQQDMMRSKLDTEVANLSVVMEEMKLVDSKHGQLIKNFTILQGPPGPRGPKGDRGSQGPPGPTGNKGQKGEKGEPGPPGPAGERGTIGPVGPPGERGSKGSKGSQGPKGSRGSPGKPGPQGPSGDPGPPGPPGKDGLPGPQGPPGFQGLQGTVGEPGVPGPRGLPGLPGVPGMPGPKGPPGPPGPSGAMEPLALQNEPTPASEVNGCPPHWKNFTDKCYYFSLEKEILEDAKLFCEDKSSHLVFINSREEQQWIKKHTVGRESHWIGLTDSEQESEWKWLDGSPVDYKNWKAGQPDNWGSGHGPGEDCAGLIYAGQWNDFQCDEINNFICEKEREAVPSSIL

SEQ ID NO: 11 - protein, 321 aa, Bos taurus
MLPLPLSILLLLTQSQSFLGEEMDVYSEKTLTDPCTLVVCAPPADSLRGHDGRDGKEGPQGEKGDPGPPGMPGPAGREGPSGRQGSMGPPGTPGPKGEPGPEGGVGAPGMPGSPGPAGLKGERGTPGPGGAIGPQGPSGAMGPPGLKGDRGDPGEKGARGETSVLEVDTLRQRMRNLEGEVQRLQNIVTQYRKAVLFPDGQAVGEKIFKTAGAVKSYSDAEQLCREAKGQLASPRSSAENEAVTQLVRAKNKHAYLSMNDISKEGKFTYPTGGSLDYSNWAPGEPNNRAKDEGPENCLEIYSDGNWNDIECREERLVICEF

SEQ ID NO: 12 - protein, 321 aa, Bos taurus; identical to SEQ ID NO: 11

SEQ ID NO: 13 - protein, 272 aa, Mus musculus
MMMRDLALAGMLISLAFLSLLPSGCPQQTTEDACSVQILVPGLKGDAGEKGDKGAPGRPGRVGPTGEKGDMGDKGQKGTVGRHGKIGPIGAKGEKGDSGDIGPPGPSGEPGIPCECSQLRKAIGEMDNQVTQLTTELKFIKNAVAGVRETESKIYLLVKEEKRYADAQLSCQARGGTLSMPKDEAANGLMASYLAQAGLARVFIGINDLEKEGAFVYSDRSPMQTFNKWRSGEPNNAYDEEDCVEMVASGGWNDVACHITMYFMCEFDKENL

SEQ ID NO: 14 - protein, 239 aa, Mus musculus
MLLLPLLPVLLCVVSVSSSGSQTCEDTLKTCSVIACGRDGRDGPKGEKGEPGQGLRGLQGPPGKLGPPGSVGSPGSPGPKGQKGDHGDNRAIEEKLANMEAEIRILKSKLQLTNKLHAFSMGKKSGKKLFVTNHEKMPFSKVKSLCTELQGTVAIPRNAEENKAIQEVATGIAFLGITDEATEGQFMYVTGGRLTYSNWKKDEPNNHGSGEDCVIILDNGLWNDISCQASFKAVCEFPA

SEQ ID NO: 15 - protein, 244 aa, Mus musculus
MSIFTSFLLLCVVTVVYAETLTEGVQNSCPVVTCSSPGLNGFPGKDGRDGAKGEKGEPGQGLRGLQGPPGKVGPTGPPGNPGLKGAVGPKGDRGDRAEFDTSEIDSEIAALRSELRALRNWVLFSLSEKVGKKYFVSSVKKMSLDRVKALCSEFQGSVATPRNAEENSAIQKVAKDIAYLGITDVRVEGSFEDLTGNRVRYTNWNDGEPNNTGDGEDCVVILGNGKWNDVPCSDSFLAICEFSD

SEQ ID NO: 16 - protein, 277 aa, Homo sapiens
MNGFASLLRRNQFILLVLFLLQIQSLGLDIDSRPTAEVCATHTISPGPKGDDGEKGDPGEEGKHGKVGRMGPKGIKGELGDMGDRGNIGKTGPIGKKGDKGEKGLLGIPGEKGKAGTVCDCGRYRKFVGQLDISIARLKTSMKFVKNVIAGIRETEEKFYYIVQEEKNYRESLTHCRIRGGMLAMPKDEAANTLIADYVAKSGFFRVFIGVNDLEREGQYMFTDNTPLQNYSNWNEGEPSDPYGHEDCVEMLSSGRWNDTECHLTMYFVCEFIKKKK

SEQ ID NO: 17 - protein, 742 aa, Homo sapiens; identical to SEQ ID NO: 8

SEQ ID NO: 18 - protein, 246 aa, Carassius auratus
LLLLQFALQLLDGAEPQNLNCPAYGGVPGTPGHNGLPGRDGRDGKDGAIGPKGEKGESGVSVQGPPGKAGPPGTAGEKGERGPSGPQGSPGSESVLESLKSEIQQLKAKIATFEKVSSVCHFRKVGQKYYITDGVVGNFDQGLKSCMEFGGTMVSPRTSAENQALLKLVVSSGLGSKKPYIGVTDRKTEGQFVDTEGKQLTFTNWGPGQPDDYKGLQDCGVIEDTGLWDDGGCGDIRPIMCEIDIK

SEQ ID NO: 19 - protein, 251 aa, Danio rerio; identical to SEQ ID NO: 7

SEQ ID NO: 20 - protein, 256 aa, Cyprinus carpio
MALFKLFLGTLLLLQFALQLLDGAEPQNLNCPAYGGVPGTPGHNGLPGRDGRDGKDGAIGPKGEKGESGVSVQGPPGKAGPPGPAGEKGERGPTGSQGSPGSESVLESLKSEIQQLKAKIATFEKVASVGHFRQVGQKYYITDGVVGTFDQGLKFCKDFGGTMVFPRTSAENQALLKLVVSSGLSSKKPYIGVTDRETEGRFVNTEGKQLTFTNWGPGQPDDYKGLQDCGVIEDSGLWDDGSCGDIRPIMCEIDNK

SEQ ID NO: 21 - protein, 222 aa, Gallus gallus
MLSYSFCMIAAAVALLTPCHAQNCAGAPELPSIPGVSGLLGLGALKRYFGSLLWPYGEEKLPECQWLQRQQDLSTSSDDELGNVLLNLRQRILQLEGVLALDGKITKVGEKIFASNGKEVNFSSALESCEETGGTLATPMNEEENKAIMGIVKQYNRYAYLGIKESDTAGQFKYVNNQPLNYTSWQQYEPNGKGTEKCVEMYTDGNWKDRKCNLYRLTVCEY

SEQ ID NO: 22 - protein, 244 aa, Mus musculus; identical to SEQ ID NO: 15

SEQ ID NO: 23 - protein, 239 aa, Mus musculus; identical to SEQ ID NO: 14

SEQ ID NO: 24 - protein, 371 aa, Bos taurus
MLLLPLSVLLLLTQPWRSLGAEMTTFSQKILANACTLVMCSPLESGLPGHDGQDGRECPHGEKGDPGSPGPAGRAGRPGWVGPIGPKGDNGFVGEPGPKGDTGPRGPPGMPGPAGREGPSGKQGSMGPPGTPGPKGETGPKGGVGAPGIQGFPGPSGLKGEKGAPGETGAPGRAGVTGPSGAIGPQGPSGARGPPGLKGDRGDPGETGAKGESGLAEVNALKQRVTILDGHLRRFQNAFSQYKKAVLFPDGQAVGEKIFKTAGAVKSYSDAEQLCREAKGQLASPRSSAENEAVTQMVRAQEKNAYLSMNDISTEGRFTYPTGEILVYSNWADGEPNNSDEGQPENCVEIFPDGKWNDVPCSKQLLVICEF

SEQ ID NO: 25 - protein, 30 aa, Gallus gallus
LLTCDKPEEKMYSCPIIQCSAPAVNGLPGD

SEQ ID NO: 26 - protein, 301 aa, Bos taurus
EEMDVYSEKTLTDPCTLVVCAPPADSLRGHDGRDGKEGPQGEKGDPGPPGMPGPAGREGPSGRQGSMGPPGTPGPKGEPGPEGGVGAPGMPGSPGPAGLKGERGAPGPGGAIGPQGPSGAMGPPGLKGDRGDPGEKGARGETSVLEVDTLRQRMRNLEGEVQRLQNIVTQYRKAVLFPDGQAVGEKIFKTAGAVKSYSDAEQLCREAKGQLASPRSSAENEAVTQLVRAKNKHAYLSMNDISKEGKFTYPTGGSLDYSNWAPGEPNNRAKDEGPENCLEIYSDGNWNDIECREERLVICEF

SEQ ID NO: 27 - protein, 116 aa, Sus scrofa
AVGEKVFSTNGQSVAFDVIRELCARAGGRIAAPRSPEENEAIASIVKKHNTYAYLGLVEGPTAGDFFYLDGTPVNYTNWYPGEPRGRGKEKCVEMYTDGQWNDRNCQQYRLAICEF

SEQ ID NO: 28 - protein, 378 aa, Sus scrofa
MLLLPLSVLILLTQPPRSLGAEMKTYSQRAVANACALVMCSPMENGLPGRDGRDGREGPRGEKGDPGLPGAVGRAGMPGLAGPVGPKGDNGSTGEPGAKGDIGPCGPPGPPGIPGPAGKEGPSGQQGNIGPPGTPGPKGETGPKGEVGALGMQGSTGARGPAGLKGERGAPGERGAPGSAGAAGPAGATGPQGPSGARGPPGLKGDRGPPGERGAKGESGLPGITALRQQVETLQGQVQRLQKAFSQYKKVELFPNGRGVGEKIFKTGGFEKTFQDAQQVCTQAGGQMASPRSETENEALSQLVTAQNKAAFLSMTDIKTEGNFTYPTGEPLVYANWAPGEPNNNGGSSGAENCVEIFPNGKWNDKACGELRLVICEF

SEQ ID NO: 29 - protein, 244 aa, Mus musculus; identical to SEQ ID NO: 15

SEQ ID NO: 30 - protein, 239 aa, Mus musculus; identical to SEQ ID NO: 14

SEQ ID NO: 31 - protein, 301 aa, Bos taurus
EEMDVYSEKTLTDPCTLVVCAPPADSLRGHDGRDGKEGPQGEKGDPGPPGMPGPAGREGPSGRQGSMGPPGTPGPKGEPGPEGGVGAPGMPGSPGPAGLKGERGAPGPGGAIGPQGPSGAMGPPGLKGDRGDPGEKGARGETSVLEVDTLRQRMRNLEGEVQRLQNIVTQYRKAVLFPDGQAVGEKIFKTAGAVKSYSDAEQLCREAKGQLASPRSSAENEAVTQLVRAKNKHAYLSMNDISKEGKFTYPTGGSLDYSNWAPGEPGNRAKDEGPENCLEIYSDGNWNDIECREERLVICEF

SEQ ID NO: 32 - protein, 2412 aa, Homo sapiens [sequence omitted]

SEQ ID NO: 33 - protein, 277 aa, Homo sapiens; identical to SEQ ID NO: 16

SEQ ID NO: 34 - protein, 238 aa, Gallus gallus
MMATSLLTTDKPEEKMYSCPIIQCSAPAVNGLPGRDGRDGPKGEKGDPGEGLRGLQGLPGKAGPQGLKGEVGPQGEKGQKGERGIVVTDDLHRQITDLEAKIRVLEDDLSRYKKALSLKDVVNIGKKMFVSTGKKYNFEKGKSLCAKAGSVLASPRNEAENTALKDLIDPSSQAYIGISDAQTEGRFMYLSGGPLTYSNWKPGEPNNHKNEDCAVIEDSGKWNDLDCSNSNIFIICEL

SEQ ID NO: 35 - protein, 30 aa, Gallus gallus; identical to SEQ ID NO: 25

SEQ ID NO: 36 - protein, 60 aa, Bos taurus
AEMTTFSQKILANACTLVMCSPLESGLPGHDGQDGRECPHGEKGDPGSPGPAGRAGRPGW

SEQ ID NO: 37 - protein, 301 aa, Bos taurus
  MISC_FEATURE (7)..(): Unknown
EEMDVYXEKTLTDPCTLVVCAPPADSLRGHDGRDGKEGPQGEKGDPGPPGMPGPAGREGPSGRQGSMGPPGTPGPKGEPGPEGGVGAPGMPGSPGPAGLKGERGAPGPGGAIGPQGPSGAMGPPGLKGDRGDPGEKGARGETSVLEVDTLRQRMRNLEGEVQRLQNIVTQYRKAVLFPDGQAVGEKIFKTAGAVKSYSDAEQLCREAKGQLASPRSSAENEAVTQLVRAKNKHAYLSMNDISKEGKFTYPTGGSLDYSNWAPGEPGNRAKDEGPENCLEIYSDGNWNDIECREERLVICEF

SEQ ID NO: 38 - protein, 244 aa, Mus musculus; identical to SEQ ID NO: 15

SEQ ID NO: 39 - protein, 239 aa, Mus musculus; identical to SEQ ID NO: 14

SEQ ID NO: 40 - protein, 15 aa, Homo sapiens
CRALIKRIQAMIPKG

SEQ ID NO: 41 - protein, 374 aa, Mus musculus
MLPFLSMLVLLVQPLGNLGAEMKSLSQRSVPNTCTLVMCSPTENGLPGRDGRDGREGPRGEKGDPGLPGPMGLSGLQGPTGPVGPKGENGSAGEPGPKGERGLSGPPGLPGIPGPAGKEGPSGKQGNIGPQGKPGPKGEAGPKGEVGAPGMQGSTGAKGSTGPKGERGAPGVQGAPGNAGAAGPAGPAGPQGAPGSRGPPGLKGDRGVPGDRGIKGESGLPDSAALRQQMEALKGKLQRLEVAFSHYQKAALFPDGRSVGDKIFRTADSEKPFEDAQEMCKQAGGQLASPRSATENAAIQQLITAHNKAAFLSMTDVGTEGKFTYPTGEPLVYSNWAPGEPNNNGGAENCVEIFTNGQWNDKACGEQRLVICEF

SEQ ID NO: 42 - protein, 248 aa, Canis familiaris
MWLRCLALALTLLMVSGIENNTKDVCVGNPGIPGTPGSHGLPGRDGRDGVKGDPGPPGPLGPPGGMPGHPGPNGMTGAPGVAGERGEKGEPGERGPPGLPASLDEELQTTLHDLRHQILQTMGVLSLHESLLVVGRKVFSSNAQSINFNDIQELCAGAGGQIAAPMSPEENEAVASIVKKYNTYAYLGLVESPDSGDFQYMDGAPVNYTNWYPGEPRGRGKEQCVEMYTDGQWNNKNCLQYRLAICEF

SEQ ID NO: 43 - protein, 375 aa, Homo sapiens
MLLFLLSALVLLTQPLGYLEAEMKTYSHRTMPSACTLVMCSSVESGLPGRDGRDGREGPRGEKGDPGLPGAAGQAGMPGQAGPVGPKGDNGSVGEPGPKGDTGPSGPPGPPGVPGPAGREGALGKQGNIGPQGKPGPKGEAGPKGEVGAPGMQGSAGARGLAGPKGERGVPGERGVPGNTGAAGSAGAMGPQGSPGARGPPGLKGDKGIPGDKGAKGESGLPDVASLRQQVEALQGQVQHLQAAFSQYKKVELFPNGQSVGEKIFKTAGFVKPFTEAQLLCTQAGGQLASPRSAAENAALQQLVVAKNEAAFLSMTDSKTEGKFTYPTGESLVYSNWAPGEPNDDGGSEDCVEIFTNGKWNDRACGEKRLVVCEF

SEQ ID NO: 44 - protein, 247 aa, Oryctolagus cuniculus
MLLLSLALTLISAPASDTCDTKDVCIGSPGIPGTPGSHGLPGRDGRDGVKGDPGPPGPMGPPGGMPGLPGRDGLIGAPGVPGERGDKGEPGERGPPGLPAYLDEELQATLHELRHHALQSIGVLSLQGSMKAVGEKIFSTNGQSVNFDAIREVCARAGGRIAVPRSLEENEAIASIVKERNTYAYLGLAEGPTAGDFYYLDGDPVNYTNWYPGEPRGQGREKCVEMYTDGKWNDKNCLQYRLVICEF

SEQ ID NO: 45 - protein, 374 aa, Mus musculus; identical to SEQ ID NO: 41

SEQ ID NO: 46 - protein, 158 aa, Homo sapiens
EAEAGSVASLRQQVEALQGQVQHLQAAFSQYKKVELFPNGQSVGEKIFKTAGFVKPFTEAQLLCTQAGGQLASPRSAAENAALQQLVVAKNEAAFLSMTDSKTEGKFTYPTGESLVYSNWAPGEPNDDGGSEDCVEIFTNGKWNDRACGEKRLVVCEF

SEQ ID NO: 47 - protein, 158 aa, Homo sapiens; identical to SEQ ID NO: 46

SEQ ID NO: 48 - protein, 158 aa, Homo sapiens; identical to SEQ ID NO: 46

SEQ ID NO: 49 - protein, 2403 aa, Homo sapiens
MGISTVILEMCLLWGQVLSTGGWIPRTTDYASLIPSEVPLDTTVAEGSPFPSELTLESTAAEGSPISLESTLESTVAEGSLIPSESTLESTVAEGSDSGLALRLVNGDGRCQGRVEILYRGSWGTVCDDSWDTNDANVVCRQLGCGWAMSAPGNAWFGQGSGPIALDDVRCSGHESYLWSCPHNGWLSHNCGHGEDAGVICSAAQPQSTLRPESWPVRISPPVPTEGSESSLALRLVNGGDRCRGRVEVLYRGSWGTVCDDYWDTNDANVVCRQLGCGWAMSAPGNAQFGQGSGPIVLDDVRCSGHESYLWSCPHNGWLTHNCGHSEDAGVICSAPLSRPTPSPDTWPTSHASTAGPESSLALRLVNGGDRCQGRVEVLYRGSWGTVCDDSWDTSDANVVCRQLGCGWATSAPGNARFGQGSGPIVLDDVRCSGYESYLWSCPHNGWLSHNCQHSEDAGVICSDTLPTITLPASTVGSESSLALRLVNGGDRCQGRVEVLYRGSWGTVCDDSWDTNDANVVCRQLGCGWAMLAPGNARFGQGSGPIVLDDVRCSGNESYLWSCPHNGWLSHNCGHSEDAGVICSGPESSLALGLVNGGDRCQGRVEVLYRGSWGTVCDDSWDTNDANVVCRQLGCGWATSAPGNARFGQGSGPIVLDDVRCSGHESYLWSCPNNGWLSHNCGHHEDAGVICSAAQSRSTPRPDTLSTITLPPSTVGSESSLTLRLVNGSDRCQGRVEVLYRGSWGTVCDDSWDTNDANVVCRQLGCGWATSAPGNARFGQGSGPIVLDDVRCSGHESYLWSCPHNGWLSHNCGHHEDAGVICSVSQSRPTPSPDTWPTSHASTAGSESSLALRLVNGGDRCQGRVEVLYRGSWGTVCDDSWDTSDANVVCRRLGCGWATSAPGNARFGQGSGPIVLDDVRCSGYESYLWSCPHNGWLSHNCQHSEDAGVICSAAHSWSTPSPDTLPTITLPASTVGSESSLALRLVNGGDRCQGRVEVLYQGSWGTVCDDSWDTNDANVVCRQLGCGWAMSAPGNARFGQGSGPIVLDDVRCSGHESYLWSCPHNGWLSHNCGHSEDAGVICSASQSRPTPSPDTWPTSHASTAGSESSLALRLVNGGDRCQGRVEVLYRGSWGTVCDDYWDTNDANVVCRQLGCGWAMSAPGNARFGQGSGPIVLDDVRCSGHESYLWSCPHDGWLSHNCGHHEDAGVICSASQSQPTPSPDTWPTSHASTAGSESSLALRLVNGGDRCQGRVEVLYRGPWGTVCDDYWDTNDANVVCRQLGCGWATSAPGNARFGQGSGPIVLDDVRCSGHESYLWSCPHNGWLSHNCGHHEDAGVICSASQSQPTPSPDTWPTSHASTAGSESSLALRLVNGGDRCQGRVEVLYRGSWGTVCDDYWDTNDANVVCRQLGCGWATSAPGSARFGQGSGPIALDDVRCSGHESYLWSCPHNGWLSHNCGHHEDAGVICSASQSQPTPSPDTWPTSRASTAGSESTLALRLVNGGDRCRGRVEVLYQGSWGTVCDDYWDTNDANVVCRQLGCGWAMSAPGNAQFGQGSGPIVLDDVRCSGHESYLWSCPHNGWLSHNCGHHEDAGVICSAAQSQSTPRPDTWLTTNLPALTVGSESSLALRLVNGGDRCRGRVEVLYRGSWGTVCDDSWDTNDANVVCRQLGCGWAMSAPGNARFGQGSGPIVLDDVRCSGNESYLWSCPHKGWLTHNCGHHEDAGVICSATQINSTTTDWWHPTTTTTARPSSNCGGFLFYASGTFSSPSYPAYYPNNAKCVWEIEVNSGYRINLGFSNLKLEAHHNCSFDYVEIFDGSLNSSLLLGKICNDTRQIFTSSYNRMTIHFRSDISFQNTGFLAWYNSFPSDATLRLVNLNSSYGLCAGRVEIYHGGTWGTVCDDSWTIQEAEVVCRQLGCGRAVSALGNAYFGSGSGPITLDDVECSGTESTLWQCRNRGWFSHNCNHREDAGVICSGNHLSTPAPFLNITRPNTDYSCGGFLSQPSGDFSSPFYPGNYPNNAKCVWDIEVQNNYRVTVIFRDVQLEGGCNYDYIEVFDGPYRSSPLIARVCDGARGSFTSSSNFMSIRFISDHSITRRGFRAEYYSSPSNDSTNLLCLPNHMQASVSRSYLQSLGFSASDLVISTWNGYYECRPQITPNLVIFTIPYSGCGTFKQADNDTIDYSNFLTAAVSGGIIKRRTDLRIHVSCRMLQNTWVDTMYIANDTIHVANNTIQVEEVQYGNFDVNISFYTSSSFLYPVTSRPYYVDLNQDLYVQAEILHSDAVLTLFVDTCVASPYSNDFTSLTYDLIRSGCVRDDTYGPYSSPSLRIARFRFRAFHFLNRFPSVYLRCKMVVCRAYDPSSRCYRGCVLRSKRDVGSYQEKVDVVLGPIQLQTPPRREEEPR

SEQ ID NO: 50 - protein, 2413 aa, Homo sapiens
MGISTVILEMCLLWGQVLSTGGWIPRTTDYASLIPSEVPLDQTVAEGSPFPSESTLESTAAEGSPISLESTLESTVAEGSLIPSESTLESTVAEGSDSGLALRLVNGDGRCQGRVEILYRGSWGTVCDDSWDTNDANVVCRQLGCGWAMSAPGNAWFGQGSGPIALDDVRCSGHESYLWSCPHNGWLSHNCGHGEDAGVICSAAQPQSTLRPESWPVRISPPVPTEGSESSLALRLVNGGDRCRGRVEVLYRGSWGTVCDDYWDTNDANVVCRQLGCGWAMSAPGNAQFGQGSGPIVLDDVRCSGHESYLWSCPHNGWLTHNCGHSEDAGVICSAPQSRPTPSPDTWPTSHASTAGPESSLALRLVNGGDRCQGRVEVLYRGSWGTVCDDSWDTSDANVVCRQLGCGWATSAPGNARFGQGSGPIVLDDVRCSGYESYLWSCPHNGWLSHNCQHSEDAGVICSAAHSWSTPSPDTLPTITLPASTVGSESSLALRLVNGGDRCQGRVEVLYRGSWGTVCDDSWDTNDANVVCRQLGCGWAMLAPGNARFGQGSGPIVLDDVRCSGNESYLWSCPHNGWLSHNCGHSEDAGVICSGPESSLALRLVNGGDRCQGRVEVLYRGSWGTVCDDSWDTNDANVVCRQLGCGWAMSAPGNARFGQGSGPIVLDDVRCSGHESYLWSCPNNGWLSHNCGHHEDAGVICSAAQSRSTPRPDTLSTITLPPSTVGSESSLTLRLVNGSDRCQGRVEVLYRGSWGTVCDDSWDTNDANVVCRQLGCGWAMSAPGNARFGQGSGPIVLDDVRCSGHESYLWSCPHNGWLSHNCGHHEDAGVICSVSQSRPTPSPDTWPTSHASTAGSESSLALRLVNGGDRCQGRVEVLYRGSWGTVCDDSWDTSDANVVCRQLGCGWATSAPGNARFGQGSGPIVLDDVRCSGYESYLWSCPHNGWLSHNCQHSEDAGVICSAAHSWSTPSPDTLPTITLPASTVGSESSLALRLVNGGDRCQGRVEVLYQGSWGTVCDDSWDTNDANVVCRQPGCGWAMSAPGNARFGQGSGPIVLDDVRCSGHESYPWSCPHNGWLSHNCGHSEDAGVICSASQSRPTPSPDTWPTSHASTAGSESSLALRLVNGGDRCQGRVEVLYRGSWGTVCDDYWDTNDANVVCRQLGCGWAMSAPGNARFGQGSGPIVLDDVRCSGHESYLWSCPHNGWLSHNCGHHEDAGVICSASQSQPTPSPDTWPTSHASTAGSESSLALRLVNGGDRCQGRVEVLYRGSWGTVCDDYWDTNDANVVCRQLGCGWATSAPGNARFGQGSGPIVLDDVRCSGHESYLWSCPHNGWLSHNCGHHEDAGVICSASQSQPTPSPDTWPTSHASTAGSESSLALRLVNGGDRCQGRVEVLYRGSWGTVCDDYWDTNDANVVCRQLGCGWATSAPGNARFGQGSGPIVLDDVRCSGHESYLWSCPHNGWLSHNCGHHEDAGVICSASQSQPTPSPDTWPTSRASTAGSESTLALRLVNGGDRCRGRVEVLYQGSWGTVCDDYWDTNDANVVCRQLGCGWAMSAPGNAQFGQGSGPIVLDDVRCSGHESYLWSCPHNGWLSHNCGHHEDAGVICSAAQSQSTPRPDTWLTTNLPALTVGSESSLALRLVNGGDRCRGRVEVLYRGSWGTVCDDSWDTNDANVVCRQLGCGWAMSAPGNARFGQGSGPIVLDDVRCSGNESYLWSCPHKGWLTHNCGHHEDAGVICSATQINSTTTDWWHPTTTTTARPSSNCGGFLFYASGTFSSPSYPAYYPNNAKCVWEIEVNSGYRINLGFSNLKLEAHHNCSFDYVEIFDGSLNSSLLLGKICNDTRQIFTSSYNRMTIHFRSDISFQNTGFLAWYNSFPSDATLRLVNLNSSYGLCAGRVEIYHGGTWGTVCDDSWTIQEAEVVCRQLGCGRAVSALGNAYFGSGSGPITLDDVECSGTESTLWQCRNRGWFSHNCNHREDAGVICSGNHLSTPAPFLNITRPNTDYSCGGFLSQPSGDFSSPFYPGNYPNNAKCVWDIEVQNNYRVTVIFRDVQLEGGCNYDYIEVFDGPYRSSPLIARVCDGARGSFTSSSNFMSIRFISDHSITRRGFRAEYYSSPSNDSTNLLCLPNHMQASVSRSYLQSLGFSASDLVISTWNGYYECRPQITPNLVIFTIPYSGCGTFKQADNDTIDYSNFLTAAVSGGIIKRRTDLRIHVSCRMLQNTWVDTMYIANDTIHVANNTIQVEEVQYGNFDVNISFYTSSSFLYPVTSRPYYVDLNQDLYVQAEILHSDAVLTLFVDTCVASPYSNDFTSLTYDLIRSGCVRDDTYGPYSSPSLRIARFRFRAFHFLNRFPSVYLRCKMVVCRAYDPSSRCYRGCVLRSKRDVGSYQEKVDVVLGPIQLQTPPRREEEPR

SEQ ID NO: 51 - protein, 1785 aa, Homo sapiens
MGISTVILEMCLLWGQVLSTGGWIPRTTDYASLIPSEVPLDQTVAEGSPFPSESTLESTAAEGSPISLESTLESTVAEGSLIPSESTLESTVAEGSDSGLALRLVNGDGRCQGRVEILYRGSWGTVCDDSWDTNDANVVCRQLGCGWAMSAPGNAWFGQGSGPIALDDVRCSGHESYLWSCPHNGWLSHNCGHGEDAGVICSAAQPQSTLRPESWPVRISPPVPTEGSESSLALRLVNGGDRCRGRVEVLYRGSWGTVCDDYWDTNDANVVCRQLGCGWAMSAPGNAQFGQGSGPIVLDDVRCSGHESYLWSCPHNGWLTHNCGHSEDAGVICSAPQSRPTPSPDTWPTSHASTAGPESSLALRLVNGGDRCQGRVEVLYRGSWGTVCDDSWDTSDANVVCRQLGCGWATSAPGNARFGQGSGPIVLDDVRCSGYESYLWSCPHNGWLSHNCQHSEDAGVICSAAHSWSTPSPDTLPTITLPASTVGSESSLALRLVNGGDRCQGRVEVLYQGSWGTVCDDSWDTNDANVVCRQPGCGWAMSAPGNARFGQGSGPIVLDDVRCSGHESYPWSCPHNGWLSHNCGHSEDAGVICSASQSRPTPSPDTWPTSHASTAGSESSLALRLVNGGDRCQGRVEVLYRGSWGTVCDDYWDTNDANVVCRQLGCGWAMSAPGNARFGQGSGPIVLDDVRCSGHESYLWSCPHNGWLSHNCGHHEDAGVICSASQSQPTPSPDTWPTSHASTAGSESSLALRLVNGGDRCQGRVEVLYRGSWGTVCDDYWDTNDANVVCRQLGCGWATSAPGNARFGQGSGPIVLDDVRCSGHESYLWSCPHNGWLSHNCGHHEDAGVICSASQSQPTPSPDTWPTSRASTAGSESTLALRLVNGGDRCRGRVEVLYQGSWGTVCDDYWDTNDANVVCRQLGCGWAMSAPGNAQFGQGSGPIVLDDVRCSGHESYLWSCPHNGWLSHNCGHHEDAGVICSAAQSQSTPRPDTWLTTNLPALTVGSESSLALRLVNGGDRCRGRVEVLYRGSWGTVCDDSWDTNDANVVCRQLGCGWAMSAPGNARFGQGSGPIVLDDVRCSGNESYLWSCPHKGWLTHNCGHHEDAGVICSATQINSTTTDWWHPTTTTTARPSSNCGGFLFYASGTFSSPSYPAYYPNNAKCVWEIEVNSGYRINLGFSNLKLEAHHNCSFDYVEIFDGSLNSSLLLGKICNDTRQIFTSSYNRMTIHFRSDISFQNTGFLAWYNSFPSDATLRLVNLNSSYGLCAGRVEIYHGGTWGTVCDDSWTIQEAEVVCRQLGCGRAVSALGNAYFGSGSGPITLDDVECSGTESTLWQCRNRGWFSHNCNHREDAGVICSGNHLSTPAPFLNITRPNTDYSCGGFLSQPSGDFSSPFYPGNYPNNAKCVWDIEVQNNYRVTVIFRDVQLEGGCNYDYIEVFDGPYRSSPLIARVCDGARGSFTSSSNFMSIRFISDHSITRRGFRAEYYSSPSNDSTNLLCLPNHMQASVSRSYLQSLGFSASDLVISTWNGYYECRPQITPNLVIFTIPYSGCGTFKQADNDTIDYSNFLTAAVSGGIIKRRTDLRIHVSCRMLQNTWVDTMYIANDTIHVANNTIQVEEVQYGNFDVNISFYTSSSFLYPVTSRPYYVDLNQDLYVQAEILHSDAVLTLFVDTCVASPYSNDFTSLTYDLIRSGCVRDDTYGPYSSPSLRIARFRFRAFHFLNRFPSVYLRCKMVVCRAYDPSSRCYRGCVLRSKRDVGSYQEKVDVVLGPIQLQTPPRREEEPR

SEQ ID NO: 52 - protein, 34 aa, Bos taurus
LIPCCPVNIKRLLIVVVVVVLLVVVIVGALLMGL

SEQ ID NO: 53 - protein, 35 aa, Canis familiaris
LGIPCFPSSLKRLLIIVVVIVLVVVVIVGALLMGL

SEQ ID NO: 54 - protein, 371 aa, Bos taurus; identical to SEQ ID NO: 24

SEQ ID NO: 55 - protein, 375 aa, Homo sapiens; identical to SEQ ID NO: 43

SEQ ID NO: 56 - protein, 197 aa, Homo sapiens
MDVGSKEVLMESPPDYSAAPRGRFGIPCCPVHLKRLLIVVVVVVLIVVVIVGALLMGLHMSQKHTEMVLEMSIGAPEAQQRLALSEHLVTTATFSIGSTGLVVYDYQQLLIAYKPAPGTCCYIMKIAPESIPSLEALNRKVHNFQMECSLQAKPAVPTSKLGQAEGRDAGSAPSGGDPAFLGMAVNTLCGEVPLYYI

SEQ ID NO: 57 - protein, 248 aa, Canis familiaris
MWLRCLALALTLLMVSGIENNTKDVCVGNPGIPGTPGSHGLPGRDGRDGVKGDPGPPGPLGPPGGMPGHPGPNGMTGAPGVAGERGEKGEPGERGPPGLPASLDEELQTTLHDLRHQILQTMGVLSLHESLLVVGRKVFSSGAQSINFNDIQELCAGAGGQIAAPMSPEENEAVASIVKKYNTYAYLGLVESPDSGDFQYMDGAPVNYTNWYPGEPRGRGKEQCVEMYTDGQWNNKNCLQYRLAICEF

SEQ ID NO: 58 - protein, 248 aa, Homo sapiens [sequence omitted]

SEQ ID NO: 59 - protein, 301 aa, Bos taurus; identical to SEQ ID NO: 26

SEQ ID NO: 60 - protein, 369 aa, Bos taurus
MLLLPLSVLLLLTQPWRSLGAEMKIYSQKTMANACTLVMCSPPEDGLPGRDGRDGREGPRGEKGDPGSPGPAGRAGMPGPAGPIGLKGDNGSAGEPGPKGDTGPPGPPGMPGPAGREGPSGKQGSMGPPGTPGPKGDTGPKGGVGAPGIQGSPGPAGLKGERGAPGDPGAPGRAGAPGPRGAIGPQGPSGARGPPGLKGDRGTPGERGAKGESGLAEVNALRQRVGILEGQLQRLQNAFSQYKKAMLFPNGRSVGEKIFKTVGSEKTFQDAQQICTQAGGQLPSPRSGAENEALTQLATAQNKAAFLSMSDTRKEGTFIYPTGEPLVYSNWAPQEPNNDGGSENCVEIFPNGKWNDKVCGEQRLVICEF

SEQ ID NO: 61 - protein, 116 aa, Sus scrofa; identical to SEQ ID NO: 27

SEQ ID NO: 62 - protein, 378 aa, Sus scrofa; identical to SEQ ID NO: 28

SEQ ID NO: 63 - protein, 34 aa, Bos taurus; identical to SEQ ID NO: 52

SEQ ID NO: 64 - protein, 369 aa, Bos taurus
MLLLPLSVLLLLTQPWRSLGAEMKIYSQKTMANACTLVMCSPPEDGLPGRDGRDGREGPRGEKGDPGSPGPAGRAGMPGPAGPIGLKGDNGSAGEPGPKGDTGPPGPPGMPGPAGREGPSGKQGSMGPPGTPGPKGDTGPKGGVGAPGIQGSPGPAGLKGERGAPGEPGAPGRAGAPGPAGAIGPQGPSGARGPPGLKGDRGTPGERGAKGESGLAEVNALRQRVGILEGQLQRLQNAFSQYKKAMLFPNGRSVGEKIFKTVGSEKTFQDAQQICTQAGGQLPSPRSGAENEALTQLATAQNKAAFLSMSDTRKEGTFIYPTGEPLVYSNWAPQEPNNDGGSENCVEIFPNGKWNDKVCGEQRLVICEF

SEQ ID NO: 65 - protein, 301 aa, Bos taurus; identical to SEQ ID NO: 31

SEQ ID NO: 66 - protein, 2412 aa, Homo sapiens [sequence omitted]

SEQ ID NO: 67 - protein, 2413 aa, Homo sapiens; identical to SEQ ID NO: 50

SEQ ID NO: 68 - protein, 374 aa, Mus musculus; identical to SEQ ID NO: 41

SEQ ID NO: 69 - protein, 35 aa, Canis familiaris; identical to SEQ ID NO: 53

SEQ ID NO: 70 - protein, 34 aa, Bos taurus; identical to SEQ ID NO: 52

SEQ ID NO: 71 - protein, 35 aa, Homo sapiens
GQSITFDAGKEQCVEMYTDGQWNDRNCLYLTICEF

SEQ ID NO: 72 - protein, 370 aa, Oryctolagus cuniculus
MAKSHLPPWLLLLLLPTLCGPGTAVWATSPLACAQGPEFWCQSLEQALQCKALGHCLQEVWGHVGADDLCQECQDIVNILTKMTKEAIFQDTIRKFLEHECDVLPLKLLVPQCHHVLDVYFPLTITYFLSQINAKAICQHLGLCQPGSPEPPLDPLPDKLVLPTLLGALPAKPGPHTQDLSAQRFPIPLPLCWLCRTLLKRIQAMIPKGVLAMAVAQVCHVVPLVVGGICQCLAERYTVILLEVLLGHVLPQLVCGLVLRCSSVDSIGQVPPTLEALPGEWLPQDPECPLCMSVTTQARNISEQTRPQAVYHACLSSQLDKQECEQFVELHTPQLLSLLSRGWDARAICQALGACVATLSPLQCIQSPHF

SEQ ID NO: 73 - protein, 247 aa, Oryctolagus cuniculus
MLLLSLALTLISAPASDTCDTKDVCIGSPGIPGTPGSHGLPGRDGRDGVKGDPGPPGPMGPPGGMPGLPGRDGLIGAPGVPGERGDKGEPGERGPPGLPAYLDEELQATLHELRHHALQSIGVLSLQGSMKAVGEKIFSTNGQSVNFDAIREVCARAGGRIAIVKEVPRSLEENEAIASRNTYAYLGLAEGPTAGDFYYLDGDPVNYTNWYPGEPRGQGREKCVEMYTDGKWNDKNCLQYRLVICEF

SEQ ID NO: 74 - protein, 369 aa, Bos taurus; identical to SEQ ID NO: 64

SEQ ID NO: 75 - protein, 301 aa, Bos taurus; identical to SEQ ID NO: 37
  MISC_FEATURE (7)..(7): Unknown

SEQ ID NO: 76 - protein, 375 aa, Homo sapiens
MLLFLLSALVLLTQPLGYLEAEMKTYSHRTTPSACTLVMCSSVESGLPGRDGRDGREGPRGEKGDPGLPGAAGQAGMPGQAGPVGPKGDNGSVGEPGPKGDTGPSGPPGPPGVPGPAGREGPLGKQGNIGPQGKPGPKGEAGPKGEVGAPGMQGSAGARGLAGPKGERGVPGERGVPGNAGAAGSAGAMGPQGSPGARGPPGLKGDKGIPGDKGAKGESGLPDVASLRQQVEALQGQVQHLQAAFSQYKKVELFPNGQSVGEKIFKTAGFVKPFTEAQLLCTQAGGQLASPRSAAENAALQQLVVAKNEAAFLSMTDSKTEGKFTYPTGESLVYSNWAPGEPNDDGGSEDCVEIFTNGKWNDRACGEKRLVVCEF

SEQ ID NO: 77 - protein, 194 aa, Rattus norvegicus
MDMGSKEVLMESPPDYSTGPRSQFRIPCCPVHLKRLLIVVVVVVLVVVVIVGALLMGLHMSQKHTEMVLEMSIGGAPETQKRLALSEHTDTIATFSIGSTGIVLYDYQRLLTAYKPAPGTYCYIMKMAPESIPSLEALARKFKNFQAKSSTPTSKLGQEEGHSAGSDSDSSGRDLAFLGLAVSTLCGVLPLYYI

SEQ ID NO: 78 - protein, 247 aa, Oryctolagus cuniculus
MLLLSLALTLISAPASDTCDTKDVCIGSPGIPGTPGSHGLPGRDGRDGVKGDPGPPAPWAPPGGMPGLPGRDGLIGAPGVPGERGDKGEPGERGPPGLPAYLDEELQATLHELRHHALQSIGVLSLQGSMKAVGEKIFSTNGQSVNFDAIREVCARAGGRIAVPRSLEENEAIASIVKERNTYAYLGLAEGPTAGDFYYLDGDPVNYTNWYPGEPRGQGREKCVEMYTDGKWNDKNCLQYRLVICEF

SEQ ID NO: 79 - protein, 652 aa, Homo sapiens; identical to SEQ ID NO: 1

SEQ ID NO: 80 - protein, 644 aa, Mus musculus
MAISTGLFLLLGLLGQPWAGAAADSQAVVCEGTACYTAHWGKLSAAEAQHRCNENGGNLATVKSEEEARHVQQALTQLLKTKAPLEAKMGKFWIGLQREKGNCTYHDLPMRGFSWVGGGEDTAYSNWYKASKSSCIFKRCVSLILDLSLTPHPSHLPKWHESPCGTPEAPGNSIEGFLCKFNFKGMCRPLALGGPGRVTYTTPFQATTSSLEAVPFASVANVACGDEAKSETHYFLCNEKTPGIFHWGSSGPLCVSPKFGCSFNNGGCQQDCFEGGDGSFRCGCRPGFRLLDDLVTCASRNPCSSNPCTGGGMCHSVPLSENYTCRCPSGYQLDSSQVHCVDIDECQDSPCAQDCVNTLGSFHCECWVGYQPSGPKEEACEDVDECAAANSPCAQGCINTDGSFYCSCKEGYIVSGEDSTQCEDIDECSDARGNPCDSLCFNTDGSFRCGCPPGWELAPNGVFCSRGTVFSELPARPPQKEDNDDRKESTMPPTEMPSSPSGSKDVSNRAQTTGLFVQSDIPTASVPLEIEIPSEVSDVWFELGTYLPTTSGHSKPTHEDSVSAHSDTDGQNLLLFYILGTVVAISLLLVLALGILIYHKRRAKKEEIKEKKPQNAADSYSWVPERAESQAPENQYSPTPGTDC

SEQ ID NO: 81 - protein, 688 aa, Homo sapiens
MWCIVLFSLLAWVYAEPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 82 - protein, 652 aa, Homo sapiens
MATSMGLLLLLLLLLTQPGAGTGADTEAVVCVGTACYTAHSGKLSAAEAQNHCNQNGGNLATVKSKEEAQHVQRVLAQLLRREAALTARMSKFWIGLQREKGKCLDPSLPLKGFSWVGGGEDTPYSNWHKELRNSCISKRCVSLLLDLSQPLLPNRLPKWSEGPCGSPGSPGSNIEGFVCKFSFKGMCRPLALGGPGQVTYTTPFQTTSSSLEAVPFASAANVACGEGDKDETQSHYFLCKEKAPDVFDWGSSGPLCVSPKYGCNFNNGGCHQDCFEGGDGSFLCGCRPGFRLLDDLVTCASRNPCSSSPCRGGATCVLGPHGKNYTCRCPQGYQLDSSQLDCVDVDECQDSPCAQECVNTPGGFRCECWVGYEPGGPGEGACQDVDECALGRSPCAQGCTNTDGSFHCSCEEGYVLAGEDGTQCQDVDECVGPGGPLCDSLCFNTQGSFHCGCLPGWVLAPNGVSCTMGPVSLGPPSGPPDEEDKGEKEGSTVPRAATASPTRGPEGTPKATPTTSRPSLSSDAPITSAPLKMLAPSGSSGVWREPSIHHATAASGPQEPAGGDSSVATQNNDGTDGQKLLLFYILGTVVAILLLLALALGLLVYRKRRAKREEKKEKKPQNAADSYSWVPERAESRAMENQYSPTPGTDC

SEQ ID NO: 83 - protein, 248 aa, Homo sapiens
MSLFPSLPLLLLSMVAASYSETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 84 - protein, 159 aa, Zephyranthes candida
MTRITFLVVAAIFLGVITPSCLSVCDSILYSGETLSAGQSLNNGPYTFIMQEDCNLVLYNVDKPIWASNTGGLARGCHLSMQRDGNLVVYSQRNRPIWASNTGGHNAANYVLILQKDRNVVIYGPAKWATGTYTGAVGISGFETGTAGIIKPVTVKSAK

SEQ ID NO: 85 - protein, 239 aa, Mus musculus; identical to SEQ ID NO: 14

SEQ ID NO: 86 - protein, 244 aa, Mus musculus; identical to SEQ ID NO: 15

SEQ ID NO: 87 - protein, 248 aa, Homo sapiens; identical to SEQ ID NO: 83

SEQ ID NO: 88 - protein, 185 aa, Mus musculus
MRLLIFLGLLWSLVATLLGSKWPEPVFGRLVSPGFPEKYADHQDRSWTLTAPPGYRLRLYFTHFDLELSYRCEYDFVKLSSGTKVLATLCGQESTDTEQAPGNDTFYSLGPSLKVTFHSDYSNEKPFTGFEAFYAAEDVDECRVSLGDSVPCDHYCHNYLGGYYCSCRAGYVLHQNKHTCSEQSL

SEQ ID NO: 89 - protein, 643 aa, Rattus norvegicus
MVTSTGLLLLLGLLGQLWAGAAADSEAVVCEGTACYTAHWGKLSAAEAQHRCNENGGNLATVKSEEEARHVQEALAQLLKTKAPSETKIGKFWIGLQREKGKCTYHDLPMKGFSWVGGGEDTTYSNWYKASKSSCISKRCVSLILDLSLKPHPSHLPKWHESPCGTPDAPGNSIEGFLCKFNFKGMCSPLALGGPGQLTYTTPFQATTSSLKAVPFASVANVVCGDEAESKTNYYLCKETTAGVFHWGSSGPLCVSPKFGCSFNNGGCQQDCFEGGDGSFRCGCRPGFRLLDDLVTCASRNPCSSNPCTGGGMCHSVPLSENYTCHCPRGYQLDSSQVHCVDIDECEDSPCDQECINTPGGFHCECWVGYQSSGSKEEACEDVDECTAAYSPCAQGCTNTDGSFYCSCKEGYIMSGEDSTQCEDIDECLGNPCDTLCINTDGSFRCGCPAGFELAPNGVSCTRGSMFSELPARPPQKEDKGDGKESTVPLTEMPGSLNGSKDVSNRAQTTDLSIQSDSSTASVPLEIEVSSEASDVWLDLGTYLPTTSGHSQPTHEDSVPAHSDSDTDGQKLLLFYILGTVVAISLLLALALGLLIYLKRKAKKEEIKEKKAQNAADSYSWIPERAESRAPENQYSPTPGTDC

SEQ ID NO: 90 - protein, 686 aa, Homo sapiens
MRLLTLLGLLCGSVATPLGPKWPEPVFGRLASPGFPGEYANDQERRWTLTAPPGYRLRLYFTHFDLELSHLCEYDFVKLSSGAKVLATLCGQESTDTERAPGKDTFYSLGSSLDITFRSDYSNEKPFTGFEAFYAAEDIDECQVAPGEAPTCDHHCHNHLGGFYCSCRAGYVLHRNKRTCSALCSGQVFTQRSGELSSPEYPRPYPKLSSCTYSISLEEGFSVILDFVESFDVETHPETLCPYDFLKIQTDREEHGPFCGKTLPHRIETKSNTVTITFVTDESGDHTGWKIHYTSTAQPCPYPMAPPNGHVSPVQAKYILKDSFSIFCETGYELLQGHLPLKSFTAVCQKDGSWDRPMPACSIVDCGPPDDLPSGRVEYITGPGVTTYKAVIQYSCEETFYTMKVNDGKYVCEADGFWTSSKGEKSLPVCEPVCGLSARTTGGRIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDF

SEQ ID NO: 91 - protein, 185 aa, Homo sapiens
MRLLTLLGLLCGSVATPLGPKWPEPVFGRLASPGFPGEYANDQERRWTLTAPPGYRLRLYFTHFDLELSHLCEYDFVKLSSGAKVLATLCGQESTDTERAPGKDTFYSLGSSLDITFRSDYSNEKPFTGFEAFYAAEDIDECQVAPGEAPTCDHHCHNHLGGFYCSCRAGYVLHRNKRTCSEQSL

SEQ ID NO: 92 - protein, 728 aa, Homo sapiens
MRWLLLYYALCFSLSKASAHTVELNNMFGQIQSPGYPDSYPSDSEVTWNITVPDGFRIKLYFMHFNLESSYLCEYDYVKVETEDQVLATFCGRETTDTEQTPGQEVVLSPGSFMSITFRSDFSNEERFTGFDAHYMAVDVDECKEREDEELSCDHYCHNYIGGYYCSCRFGYILHTDNRTCRVECSDNLFTQRTGVITSPDFPNPYPKSSECLYTIELEEGFMVNLQFEDIFDIEDHPEVPCPYDYIKIKVGPKVLGPFCGEKAPEPISTQSHSVLILFHSDNSGENRGWRLSYRAAGNECPELQPPVHGKIEPSQAKYFFKDQVLVSCDTGYKVLKDNVEMDTFQIECLKDGTWSNKIPTCKIVDCRAPGELEHGLITFSTRNNLTTYKSEIKYSCQEPYYKMLNNNTGIYTCSAQGVWMNKVLGRSLPTCLPECGQPSRSLPSLVKRIIGGRNAEPGLFPWQALIVVEDTSRVPNDKWFGSGALLSASWILTAAHVLRSQRRDTTVIPVSKEHVTVYLGLHDVRDKSGAVNSSAARVVLHPDFNIQNYNHDIALVQLQEPVPLGPHVMPVCLPRLEPEGPAPHMLGLVAGWGISNPNVTVDEIISSGTRTLSDVLQYVKLPVVPHAECKTSYESRSGNYSVTENMFCAGYYEGGKDTCLGDSGGAFVIFDDLSQRWVVQGLVSWGGPEECGSKQVYGVYTKVSNYVDWVWEQMGLPQSVVEPQVER

SEQ ID NO: 93 - protein, 699 aa, Homo sapiens
MRWLLLYYALCFSLSKASAHTVELNNMFGQIQSPGYPDSYPSDSEVTWNITVPDGFRIKLYFMHFNLESSYLCEYDYVKVETEDQVLATFCGRETTDTEQTPGQEVVLSPGSFMSITFRSDFSNEERFTGFDAHYMAVDVDECKEREDEELSCDHYCHNYIGGYYCSCRFGYILHTDNRTCRVECSDNLFTQRTGVITSPDFPNPYPKSSECLYTIELEEGFMVNLQFEDIFDIEDHPEVPCPYDYIKIKVGPKVLGPFCGEKAPEPISTQSHSVLILFHSDNSGENRGWRLSYRAAGNECPELQPPVHGKIEPSQAKYFFKDQVLVSCDTGYKVLKDNVEMDTFQIECLKDGTWSNKIPTCKIVDCRAPGELEHGLITFSTRNNLTTYKSEIKYSCQEPYYKMLNNNTGIYTCSAQGVWMNKVLGRSLPTCLPVCGLPKFSRKLMARIFNGRPAQKGTTPWIAMLSHLNGQPFCGGSLLGSSWIVTAAHCLHQSLDPEDPTLRDSDLLSPSDFKIILGKHWRLRSDENEQHLGVKHTTLHPQYDPNTFENDVALVELLESPVLNAFVMPICLPEGPQQEGAMVIVSGWGKQFLQRFPETLMEIEIPIVDHSTCQKAYAPLKKKVTRDMICAGEKEGGKDACAGDSGGPMVTLNRERGQWYLVGTVSWGDDCGKKDRYGVYSYIHHNKDWIQRVTGVRN

SEQ ID NO: 94 - protein, 239 aa, Mus musculus
  MISC_FEATURE (91)..(116): Unknown
MLLLPLLPVLLCVVSVSSSGSQTCEDTLKTCSVIACGRDGRDGPKGEKGEPGQGLRGLQGPPGKLGPPGSVGSPGSPGPKGQKGDHGDNRXXXXXXXXXXXXXXXXXXXXXXXXXXHAFSMGKKSGKKLFVTNHEKMPFSKVKSLCTELQGTVAIPRNAEENKAIQEVATGIAFLGITDEATEGQFMYVTGGRLTYSNWKKDEPNNHGSGEDCVIILDNGLWNDISCQASFKAVCEFPA

SEQ ID NO: 95 - protein, 185 aa, Oncorhynchus mykiss
MEKLAILLLLSASIALGDANLTQLLGLEPLLKTKVEQTTPEAQVEAVQEGIKEGSCPSDWYTYGSHCFKFVSIQQSFVDSEQNCLALGGNLASVHSLLEYQFMQALTKDANGHLHSTWLGGFDAIKEGTWMWSDGSRFDYTNWDTDEPNNAGEGEDCLHMNAASAKLWFDVPCEWKFASLCSRRM

SEQ ID NO: 96 - protein, 240 aa, Sus scrofa
MSLFPSLHLLLLIVMTASHTETENCEDIQNTCLVISCDSPGINGLPGKDGLDGAKGEKGEPGQGLIGLQGLPGMVGPQGSPGIPGLPGLKGQKGDSGIDPGNSLANLRSELDNIKKWLIFAQGKQVGKKLYLTNGKKMSFNGVKALCAQFQASVATPTNSRENQAIQELAGTEAFLGITDEYTEGQFVDLTGKRVRYQNWNDGEPNNADSAEHCVEILKDGKWNDIFCSSQLSAVCEFPA

SEQ ID NO: 97 - protein, 239 aa, Mus musculus; identical to SEQ ID NO: 14

SEQ ID NO: 98 - protein, 244 aa, Mus musculus; identical to SEQ ID NO: 15

SEQ ID NO: 99 - protein, 381 aa, Macaca nemestrina
MSDSKEPRLQQLDLLEEEQLGGVGFRQTRGYKSLAGCLGHGPLVLQLLSFTLLAGLLVQVSKVPSSLSQGQSKQDAIYQNLTQLKVAVSELSEKSKQQEIYQELTRLKAAVGELPEKSKQQEIYEELTRLRAAVGELPEKSKLQEIYQELTRLKAAVGELPEKSKQQEIYQELSRLKAAVGDLPEKSKQQEIYQKLTQLKAAVDGLPDRSKQQEIYQELIQLKAAVERLCHPCPWEWTFFQGNCYFMSNSQRNWHDSITACQEVGAQLVVIKSAEEQNFLQLQSSRSNRFTWMGLSDLNHEGTWQWVDGSPLLPSFKQYWNKGEPNNVGEEDCAEFSGNGWNDDKCNLAKFWICKKSAASCSGDEERLLSPAPTTPNPPPA

SEQ ID NO: 100 - protein, 246 aa, Carassius auratus; identical to SEQ ID NO: 18

SEQ ID NO: 101 - protein, 251 aa, Danio rerio; identical to SEQ ID NO: 7

SEQ ID NO: 102 - protein, 256 aa, Cyprinus carpio; identical to SEQ ID NO: 20

SEQ ID NO: 103 - protein, 31 aa, Sus scrofa
TKGEKGEPGPGFRGSQGPPGKMGPPGNIGET

SEQ ID NO: 104 - protein, 254 aa, Gallus gallus
MTLLQPFSALLLCLSLMMATSLLTTDKPEEKMYSCPIIQCSAPAVNGLPGRDGRDGPKGEKGDPGEGLRGLQGLPGKAGPQGLKGEVGPQGEKGQKGERGIVVTDDLHRQITDLEAKIRVLEDDLSRYKKALSLKDVVNVGKKMFVSTGKKYNFEKGKSLCAKAGSVLASPRNEAENTALKDLIDPSSQAYIGISDAQTEGRFMYLSGGPLTYSNWKPGEPNNHKNEDCAVIEDSGKWNDLDCSNSNIFIICEL

SEQ ID NO: 105 - protein, 238 aa, Rattus norvegicus
MLLLPLLVLLCVVSVSSSGSQTCEETLKTCSVIACGRDGRDGPKGEKGEPGQGLRGLQGPPGKLGPPGSVGAPGSQGPKGQKGDRGDSRAIEVKLANMEAEINTLKSKLELTNKLHAFSMGKKSGKKFFVTNHERMPFSKVKALCSELRGTVAIPRNAEENKAIQEVAKTSAFLGITDEVTEGQFMYVTGGRLTYSNWKKDEPNDHGSGEDCVTIVDNGLWNDISCQASHTAVCEFPA

SEQ ID NO: 106 - protein, 244 aa, Rattus norvegicus
MSLFTSFLLLCVLTAVYAETLTEGAQSSCPVIACSSPGLNGFPGKDGHDGAKGEKGEPGQGLRGLQGPPGKVGPAGPPGNPGSKGATGPKGDRGESVEFDTTNIDLEIAALRSELRAMRKWVLLSMSENVGKKYFMSSVRRMPLNRAKALCSELQGTVATPRNAEENRAIQNVAKDVAFLGITDQRTENVFEDLTGNRVRYTNWNEGEPNNVGSGENCVVLLTNGKWNDVPCSDSFLVVCEFSD

SEQ ID NO: 107 - protein, 248 aa, Homo sapiens; identical to SEQ ID NO: 83

SEQ ID NO: 108 - protein, 329 aa, Homo sapiens
MWCIVLFSLLAWVYAEPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVE

SEQ ID NO: 109 - protein, 109 aa, Aloe arborescens
DNILYSSEVLHENQYISYGPYEFIMQHDCNLVLYESGNPTWASNTGGLALHCRATLQTDGNLVVQNSANRIIWQSNTGTGTNGDYLLVLQKNGNVVIVGPPIWATGTGR

SEQ ID NO: 110 - protein, 248 aa, Homo sapiens
MSLFPSLPLLLLSMVAASYSETVTCEDAQKTCPAVIACSSPGINGFPGKDGCDGTKGEKGEPGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 111 - protein, 248 aa, Homo sapiens; identical to SEQ ID NO: 83

SEQ ID NO: 112 - protein, 248 aa, Homo sapiens; identical to SEQ ID NO: 83

SEQ ID NO: 113 - protein, 248 aa, Homo sapiens [sequence omitted]

SEQ ID NO: 114 - protein, 248 aa, Homo sapiens
MSLFPSLPLLLLSMVAASYSETVTCEDAQKTCPAVIACSSPGINGFPGKDGRDGTKEEKGEPGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 115 - protein, 248 aa, Homo sapiens; identical to SEQ ID NO: 83

SEQ ID NO: 116 - protein, 248 aa, Homo sapiens; identical to SEQ ID NO: 83

SEQ ID NO: 117 - protein, 652 aa, Homo sapiens; identical to SEQ ID NO: 82

SEQ ID NO: 118 - protein, 299 aa, Homo sapiens [sequence omitted]

SEQ ID NO: 119 - protein, 169 aa, Homo sapiens
MGPALLALSFLWTMALTEDTCPAMLEYVALNSEPGMASKNPSRRHGLSLLVVDQGPGARGVRTDQGPSGADPGSLELHGECPIFPSEQVILTHHNNYPFSTEDQDNDRDAENCAVHYQGAWWYASCHLSHLNGVYLGGARDSFTNGINWKSGKGNNYSYKVSEMKVRPT

SEQ ID NO: 120 - protein, 326 aa, Homo sapiens
MELSGATMARGLAVLLVLFLHIKNLPAQAADTCPEVKVVGLEGSDKLTILRGCPGLPGAPGPKGEAGVIGERGERGLPGAPGKAGPVGPKGDRGEKGMRGEKGDAGQSQSCATGPRNCKDLLDRGYFLSGWHTIYLPDCRPLTVLCDMDTDGGGWTVFQRRMDGSVDFYRDWAAYKQGFGSQLGEFWLGNDNIHALTAQGSSELRVDLVDFEGNHQFAKYKSFKVADEAEKYKLVLGAFVGGSAGNSLTGHNNNFFSTKDQDNDVSSSNCAEKFQGAWWYADCHASNLNGLYLMGPHESYANGINWSAAKGYKYSYKVSEMKVRPA

SEQ ID NO: 121 - protein, 299 aa, Homo sapiens [sequence omitted]

SEQ ID NO: 122 - protein, 314 aa, Mus musculus
MALGSAALFVLTLTVHAAGTCPELKVLDLEGYKQLTILQGCPGLPGAAGPKGEAGAKGDRGESGLPGIPGKEGPTGPKGNQGEKGIRGEKGDSGPSQSCATGPRTCKELLTQGHFLTGWYTIYLPDCRPLTVLCDMDTDGGGWTVFQRRLDGSVDFFRDWTSYKRGFGSQLGEFWLGNDNIHALTTQGTSELRVDLSDFEGKHDFAKYSSFQIQGEAEKYKLILGNFLGGGAGDSLTPHNNRLFSTKDQDNDGSTSSCAMGYHGAWWYSQCHTSNLNGLYLRGPHKSYANGVNWKSWRGYNYSCKVSEMKVRLI

SEQ ID NO: 123 - protein, 102 aa, Homo sapiens
MELDRAVGVLGAATLLLSFLGMAWALQAADTCPEVKMVGLEGSDKLTILRGCPGLPGAPGDKGEAGTNGKRGERGPPGPPGKAGPPGPNGAPGEPQPCLTGD

SEQ ID NO: 124 - protein, 182 aa, Homo sapiens
MELDRAVGVLGAATLLLSFLGMAWALQAADTCPEVKMVGLEGSDKLTILRGCPGLPGAPGDKGEAGTNGKRGERGPPGPPGKAGPPGPNGAPGEPQPCLTGPRTCKDLLDRGHFLSGWHTIYLPDCRPLTVLCDMDTDGGGWTVSVGLGRGGQPGSPGGQAAHLVGEHTLEFSILLVGDSQR

SEQ ID NO: 125 - protein, 275 aa, Homo sapiens
MELDRAVGVLGAATLLLSFLGMAWALQAADTCPGERGPPGPPGKAGPPGPNGAPGEPQPCLTGPRTCKDLLDRGHFLSGWHTIYLPDCRPLTVLCDMDTDGGGWTVFQRRVDGSVDFYRDWATYKQGFGSRLGEFWLGNDNIHALTAQGTSELRVDLVDFEDNYQFAKYRSFKVADEAEKYNLVLGAFVEGSAGDSLTFHNNQSFSTKDQDNDLNTGNCAVMFQGAWWYKNCHVSNLNGRYLRGTHGSFANGINWKSGKGYNYSYKVSEMKVRPA

SEQ ID NO: 126 - protein, 326 aa, Homo sapiens; identical to SEQ ID NO: 120

SEQ ID NO: 127 - protein, 313 aa, Homo sapiens
MELDRAVGVLGAATLLLSFLGMAWALQAADTCPEVKMVGLEGSDKLTILRGCPGLPGAPGDKGEAGTNGKRGERGPPGPPGKAGPPGPNGAPGEPQPCLTGPRTCKDLLDRGHFLSGWHTIYLPDCRPLTVLCDMDTDGGGWTVFQRRVDGSVDFYRDWATYKQGFGSRLGEFWLGNDNIHALTAQGTSELRVDLVDFEDNYQFAKYRSFKVADEAEKYNLVLGAFVEGSAGDSLTFHNNQSFSTKDQDNDLNTGNCAVMFQGAWWYKNCHVSNLNGRYLRGTHGSFANGINWKSGKGYNYSYKVSEMKVRPA

SEQ ID NO: 128 - protein, 335 aa, Rattus norvegicus
MWWPMLWAFPVLLCLCSSQALGQESGACPDVKIVGLGAQDKVAVIQSCPSFPGPPGPKGEPGSPAGRGERGLQGSPGKMGPPGSKGEPGTMGPPGVKGEKGERGTASPLGQKELGDALCRRGPRSCKDLLTRGIFLTGWYTIYLPDCRPLTVLCDMDVDGGGWTVFQRRVDGSINFYRDWDSYKRGFGNLGTEFWLGNDYLHLLTANGNQELRVDLREFQGQTSFAKYSSFQVSGEQEKYKLTLGQFLEGTAGDSLTKHNNMAFSTHDQDNDTNGGKNCAALFHGAWWYHDCHQSNLNGRYLPGSHESYADGINWLSGRGHRYSYKVAEMKIRAS

SEQ ID NO: 129 - protein, 313 aa, Homo sapiens; identical to SEQ ID NO: 127

SEQ ID NO: 130 - protein, 306 aa, Mus musculus
LGSAALFVLTLTVHAAGTCPELKVLDLEGYKQLTILQGCPGLPGAAGPKGEAGAKGDRGESGLPGIPGKEGPTGPKGNQGEKGIRGEKGDSGPSQSCATGPRTCKELLTQGHFLTGWYTIYLPDCRPMTVLCDMDTDGGGWTVFQRRLDGSVDFFRDWTSYKRGFGSQLGEFWLGNDNIHALTTQGTSELRVDLSDFEGKHDFAKYSSFQIQGEAEKYKLILGNFLGGGAGDSLTPHNNRLFSTKDQDNDGSTSSCAMGYHGAWWYSQCHTSNLNGLYLRGPHKSYANGVNWKSWRGYNYSCKVSE

SEQ ID NO: 131 - protein, 334 aa, Mus musculus
MQWPTLWAFSGLLCLCPSQALGQERGACPDVKVVGLGAQDKVVVIQSCPGFPGPPGPKGEPGSPAGRGERGFQGSPGKMGPAGSKGEPGTMGPPGVKGEKGDTGAAPSLGEKELGDTLCQRGPRSCKDLLTRGIFLTGWYTIHLPDCRPLTVLCDMDVDGGGWTVFQRRVDGSIDFFRDWDSYKRGFGNLGTEFWLGNDYLHLLTANGNQELRVDLQDFQGKGSYAKYSSFQVSEEQEKYKLTLGQFLEGTAGDSLTKHNNMSFTTHDQDNDANSMNCAALFHGAWWYHNCHQSNLNGRYLSGSHESYADGINWGTGQGHHYSYKVAEMKIRAS

SEQ ID NO: 132 - protein, 319 aa, Rattus norvegicus
MVLGSAALFVLSLCVTELTLHAADTCPEVKVLDLEGSNKLTILQGCPGLPGALGPKGEAGAKGDRGESGLPGHPGKAGPTGPKGDRGEKGVRGEKGDTGPSQSCATGPRTCKELLTRGYFLTGWYTIYLPDCRPLTVLCDMDTDGGGWTVFQRRIDGTVDFFRDWTSYKQGFGSQLGEFWLGNDNIHALTTQGTNELRVDLADFDGNHDFAKYSSFQIQGEAEKYKLILGNFLGGGAGDSLTSQNNMLFSTKDQDNDQGSSNCAVRYHGAWWYSDCHTSNLNGLYLRGLHKSYANGVNWKSWKGYNYSYKVSEMKVRLI

SEQ ID NO: 133 - protein, 334 aa, Mus musculus; identical to SEQ ID NO: 131

SEQ ID NO: 134 - protein, 326 aa, Homo sapiens [sequence omitted]

SEQ ID NO: 135 - protein, 323 aa, Sus scrofa
MDTRGVAAAMRPLVLLVAFLCTAAPALDTCPEVKVVGLEGSDKLSILRGCPGLPGAAGPKGEAGASGPKGGQGPPGAPGEPGPPGPKGDRGEKGEPGPKGESWETEQCLTGPRTCKELLTRGHILSGWHTIYLPDCQPLTVLCDMDTDGGGWTVFQRRSDGSVDFYRDWAAYKRGFGSQLGEFWLGNDHIHALTAQGTNELRVDLVDFEGNHQFAKYRSFQVADEAEKYMLVLGAFVEGNAGDSLTSHNNSLFTTKDQDNDQYASNCAVLYQGAWWYNSCHVSNLNGRYLGGSHGSFANGVNWSSGKGYNYSYKVSEMKFRAT

SEQ ID NO: 136 - protein, 319 aa, Homo sapiens
MARGLAVLLVLFLHIKNLPAQAADTCPEVKVVGLEGSDKLTILRGCPGLPGAPGPKGEAGVIGERGERGLPGAPGKAGPVGPKGDRGEKGMRGEKGDAGQSQSCATGPRNCKDLLDRGYFLSGWHTIYLPDCRPLTVLCDMDTDGGGWTVFQRRMDGSVDFYRDWAAYKQGFGSQLGEFWLGNDNIHALTAQGSSELRVDLVDFEGNHQFAKYKSFKVADEAEKYKLVLGAFVGGSAGNSLTGHNNNFFSTKDQDNDVSSSNCAEKFQGAWWYADCHASNLNGLYLMGPHESYANGINWSAAKGYKYSYKVSEMKVRPA

SEQ ID NO: 137 - protein, 187 aa, Drosophila melanogaster
MKSCFFVLFLWTLLFEVGQSSPHTCPSGSPNGIHQLMLPEEEPFQVTQCKTTARDWIVIQRRLDGSVNFNQSWFSYKDGFGDPNGEFFIGLQKLYLMTREQPHELFIQLKHGPGATVYAHFDDFQVDSETELYKLERVGKYSGTAGDSLRYHINKRFSTFDRDNDESSKNCAAEHGGGWWFHSCLSR

(19) United States
(12) Patent Application Publication
(10) Pub. No.: US 2005/0137126 A1
(43) Pub. Date: Jun. 23, 2005

(54) Treatment of SARS in individuals

(75) Inventors: Leif Kongerslev, Birkerod (DK); Dietmar Weilguny, Virum (DK); Finn Matthiesen, Bronshoj (DK); Jens Christian Jensenius, Odense M (DK)

Correspondence Address: BROWDY AND NEIMARK, P.L.L.C.;624 NINTH STREET, NW, SUITE 300, WASHINGTON, DC, 20001-5303, US

(73) Assignees: Natlmmune A/S, Copenhagen (DK); Aarhus Universitet, Aarhus (DK)

(21) Appl. No.: 10/820,155

(22) Filed: Apr. 8, 2004

(30) Foreign Application Priority Data
Apr. 9, 2003 (DK) PA 2003 00549
May 10, 2003 (DK) PA 2003 00714

(51) Int. Cl. A61K 38/16

(52) U.S. Cl. 514/8

(57) ABSTRACT

The present invention pertains to the use of subunits and oligomers of collectins and/or ficolins, such as mannan-binding lectin (MBL) in prophylactic and/or curative treatment of Severe Acute Respiratory Syndrome (SARS) in an individual.

The present invention pertains to the use of subunits and oligomers of collectins and/or ficolins, such as mannan-binding lectin (MBL) in prophylactic and/or curative treatment of Severe Acute Respiratory Syndrome (SARS) in an individual.

SARS infection presents the symptoms of high fever, dry cough, myalgin (muscle soreness) and sore throat. Most individuals suffering from SARS develop breathing difficulties eventually requiring ventilator support, and severe thrombocytopenia. 5-10 percent of individuals suffering from SARS will eventually die due to the disease.

The cause of SARS is not yet known. It has been speculated that SARS may be caused by a virus and among these, coronaviruses and paramyxoviruses have been mentioned.

Symptoms of SARS seem to start 2-14 days after exposure.

### SUMMARY OF THE INVENTION

By the present invention treatment and/or prophylaxis of Severe Acute Respiratory Syndrome using collectins and/or ficolins is suggested.

Collectins all exhibit the following architecture: they have an N-terminal cysteine-rich region that appears to form inter-chain disulfide bonds, followed by a collagen-like region, an a-helical coiled-coil region and finally a C-type lectin domain which is the pattern-recognizing region and is referred to as the carbohydrate recognition domain (CRD). The name collectin is derived from the presence of both collagen and lectin domains. The a-helical coiled-coil region initiates trimerisation of the individual polypetides to form collagen triple coils, thereby generating collectin subunits each consisting of 3 individual polypeptides, whereas the N-terminal region mediates formation of oligomers of subunits. Different collectins exhibit distinctive higher order structures, typically either tetramers of subunits or hexamers of subunits. The grouping of large numbers of binding domains allows collectins to bind with high avidity to microbial cell walls, despite a relatively low intrinsic affinity of each individual CRD for carbohydrates.

C-type CRDs are found in proteins with a widespread occurrence, both in phylogenetic and functional perspective. The different CRDs of the different collectins enable them to recognise a range of distinct microbial surface components exposed on different microorganisms. The terminal CRDs are distributed in such a way that all three domain target surfaces that present binding sites has a spacing of approximately 53 Å (Sheriff et al., 1994; Weis & Drickamer, 1994). This property of ‘pattern recognition’ may contribute further to the selectively binding of microbial surfaces. The collagenous region or possibly the N-terminal tails of the collecting, are recognised by specific receptors on phagocytes, and is the binding site for associated proteases that are activated to initiate the complement cascade upon binding of the CRD domain to a target.

Ficolins, like MBL, are lectins that contain a collagen-like domain. Unlike MBL, however, they have a fibrinogen-like domain, which is similar to fibrinogen β- and γ-chains. Ficolins also forms oligomers of structural subunits, each of which is composed of three identical 35 kDa polypeptides. Each subunit is composed of an amino-terminal, cysteine-rich region; a collagen-like domain that consists of tandem repeats of Gly-Xaa-Yaa triplet sequences (where Xaa and Yaa represent any amino acid); a neck region; and a fibrinogen-like domain. The oligomers of ficolins comprises two or more subunits, especially a tetrameric form of ficolin has been observed.

Some of the ficolins triggers the activation of the complement system substantially in similar way as done by MBL. This triggering of the complement system results in the activation of novel serine proteases (MASPs) as described above.

The fibrinogen-like domain of several lectins has a similar function to the CRD of C-type lectins including MBL, and hereby function as pattern-recognition receptors to discriminate pathogens from self.

Serum ficolins have a common binding specificity for GlcNAc (N-acetyl-glucosamine), elastin or GalNAc (N-acetyl-galactosamine). The fibrinogen-like domain is responsible for the carbohydrate binding. In human serum, two types of ficolin, known as L-ficolin (P35, ficolin L, ficolin 2 or hucolin) and H-ficolin (Hakata antigen, ficolin 3 or thermolabile b2-macroglycoprotein), have been identified, and both of them have lectin activity. L-ficolin recognises GlcNAc and H-ficolin recognises GalNAc. Another ficolin known as M-ficolin (P35-related protein, Ficolin 1 or Ficolin A) is not considered to be a serum protein and is found in leucocytes and in the lungs. L-ficolin and H-ficolin activate the lectin-complement pathway in association with MASPs. M-Ficolin, L-ficolin and H-ficolin has calcium-independent lectin activity.

Mannan-binding lectin (MBL), synonymous to mannose-binding lectin, mannan-binding protein or mannose-binding protein (MBP), belongs to a subgroup of C-type lectins, termed collecting, since these soluble proteins are composed of subunits presenting three CRDs attached to a collagenous stalk². MBL interact with carbohydrates presented by a wide range of micro-organisms and accumulating evidence shows that it plays an important role in the innate immune defence³. When bound to carbohydrate MBL is able to activate the complement system.

The complement system may be activated via three different pathways: the classical pathway, the alternative pathway, and the newly described third pathway, the mannan-binding lectin (MBL) pathway which is initiated by the binding of MBL to carbohydrates presented by micro-organisms. The components of the alternative pathway and of the MBL pathway are parts of the innate immune defence, also termed the natural or the non-clonal, immune defence, while the classical pathway involves cooperation with antibodies of the specific immune defence⁴.

The human MBL protein is composed of up to 18 identical 32 kDa polypeptide chains²⁷, each comprising a short N-terminal segment of 21 amino acids including three cysteine residues, followed by 7 repeats of the collagenous motif Gly-X-Y interrupted by a Gln residues followed by another 12 Gly-X-Y repeats. A small 34 residue ‘neck-region’ joins the C-terminal Ca²⁺-dependent lectin domain of 93 amino acids with the collagenous part of the molecule²⁸.

The collagenous regions of the three polypeptide chains combine to form a subunit which is stabilised covalently by disulphide bridges. Individual subunits are joined by disulphide bridges as well as by non-covalently interactions²⁷.

The concentration of MBL in human serum is largely genetically determined, but reportedly increases up to threefold during acute phase reactions⁸. Three mutations causing structural alterations and two mutations in the promotor region are associated with MBL deficiency⁹.

A wide range of oligosaccharides can bind to MBL. As the target sugars are not normally exposed on mammalian cell surfaces at high densities, MBL does not usually recognize self-determinants, but is particularly well suited to interactions with microbial cell surfaces presenting repetitive carbohydrate determinants.

Thus, the invention features the use of MBL, purified from natural sources or from material produced by recombinant technologies, or by any other suitable MBL-producing cell line, for the prophylaxis and/or treatment of SARS. The MBL may be given before or after start of the medical treatment and for any duration of time deemed suitable.

MBL is believed to exert its anti-SARS activity mainly through its opsonizing activity (preparation of microorganisms for phagocytosis). This activity is dependent on activation of complement after binding of MBL to the microbial surface and deposition of C4b and C3b on the microorganism. MBL can also promote the direct complement-mediated killing of the microorganism through the activation of the terminal lytic pathway of complement and insertion of the membrane attack complex (MAC) in the membrane. This mechanism is considered of minor importance.

It is possible according to the invention to treat SARS prophylactically. By prophylactic treatment with MBL it is possible to prevent subsequent SARS or to reduce the risk of the individual contracting SARS.

In another aspect the present invention is related to the use of a composition comprising at least one mannan-binding lectin (MBL) subunit, or at least one oligomer comprising the at least one mannan-binding lectin (MBL) subunit, in the manufacture of a medicament for prophylactic, ameliorating or curative treatment of SARS in an individual initially having low plasma levels of MBL, such as plasma levels of about 0 mg/ml, or plasma levels in excess of 10 ng/. In particular the individual may be genetically disposed to an MBL deficiency or have acquired an MBL deficiency leading to an increased risk of suffering from infections. Accordingly, the invention also concerns treatment of SARS in individuals suffering from a mannan-binding lectin (MBL) deficiency including any deficiency in the production of MBL and/or function of MBL, in particular however, individuals who have or are suspected to have SARS.

In yet another aspect there is provided a method for estimating the probability of the occurrence of any severe outcome of SARS in an individual, said method comprising the step of measuring the concentration of MBL in plasma or serum obtained from the individual, and estimating the probability on the basis of the measured concentration.

Also, by genotyping the individuals in question it is possible to estimate the probability.

### DETAILED DESCRIPTION OF THE INVENTION

SARS may be prevented and/or treated in individuals independent on their serum collectin and/or ficolin level, such as MBL level.

The collectin according to the invention may be any collectin capable of preventing or treating SARS in an individual.

Accordingly, the collectin may be selected from the group consisting of MBL (mannose-binding lectin), SP-A (lung surfactant protein A), SP-D (lung surfactant protein D), BK (or BC, bovine conglutinin), CL-L1 (Ohtani et al. 1999, Molecular cloning of a novel human collectin from liver (CL-L1), J. Biol. Chem. 274:13681-89), CL-P1 (Ohtani et al. 2001. The membrane-type collectin CL-P1 is a scavenger receptor on vascular endothelial cells. J. Biol. Chem. 276:44222-28), and CL43 (collectin-43). Most preferably the collectin is MBL. (Holmskov et al. 2003, Annu Rev. Immunol. 21:547-78).

In a particular preferred embodiment the collectin has one of the sequences listed below with reference to their database and accession No.

Collectins

1: Q9NPY3
Complement component C1q receptor precursor (Complement component 1, q
subcomponent, receptor 1) (C1qRp) (C1qR(p)) (C1q/MBL/SPA receptor) (CD93
antigen) (CDw93)
gi|21759074|sp|Q9NPY3|CD93_HUMAN[21759074]
2: BAC05523
collectin placenta 1 [Mus musculus]
gi|21901969|dbj|BAC05523.1|[21901969]
3: AAM34743
46-kDa collectin precursor [Bos taurus]
gi|21105687|gb|AAM34743.1|AF509590_1[21105687]
4: AAM34742
46-kDa collectin precursor [Bos taurus]
gi|21105685|gb|AAM34742.1|AF509589_1[21105685]
5: XP_139613
similar to collectin sub-family member 10; collectin liver 1; collectin 34 [Mus
musculus]
gi|20903807|ref|XP_139613.1|[20903807]
6: XP_123211
similar to collectin sub-family member 12 [Mus musculus]
gi|20876566|ref|XP_123211.1|[20876566]
7: NP_571645
mannose binding-like lectin [Danio rerio]
gi|18858997|ref|NP_571645.1|[18858997]
8: NP_569057
collectin sub-family member 12, isoform I; scavenger receptor with C-type
lectin; collectin placenta 1 [Homo sapiens]
gi|18641360|ref|NP_569057.1|[18641360]
9: NP_110408
collectin sub-family member 12, isoform II; scavenger receptor with C-type
lectin; collectin placenta 1 [Homo sapiens]
gi|18641358|ref|NP_110408.2|[18641358]
10: NP_569716
collectin sub-family member 12 [Mus musculus]
gi|18485494|ref|NP_569716.1|[18485494]
11: AAL61856
43 kDa collectin precursor [Bos taurus]
gi|18252111|gb|AAL61856.1|[18252111]
12: AAL61855
43 kDa collectin precursor [Bos taurus]
gi|18252109|gb|AAL61855.1|[18252109]
13: BAB22581
data source: SPTR, source key: Q9Y6Z7, evidence: ISS˜homolog to COLLECTIN
34˜putative [Mus musculus]
gi|12833584|dbj|BAB22581.1|[12833584]
14: NP_034905
mannose binding lectin, liver (A) [Mus musculus]
gi|6754654|ref|NP_034905.1|[6754654]
15: NP_034906
mannose binding lectin, serum (C) [Mus musculus]
gi|6754656|ref|NP_034906.1|[6754656]
16: NP_006429
collectin sub-family member 10; collectin liver 1; collectin 34 [Homo sapiens]
gi|5453619|ref|NP_006429.1|[5453619]
17: BAB72147
collectin placenta 1 [Homo sapiens]
gi|17026101|dbj|BAB72147.1|[17026101]
18: AAF63470
mannose binding-like lectin precursor [Carassius auratus]
gi|7542474|gb|AAF63470.1|AF227739_1[7542474]
19: AAF63469
mannose binding-like lectin precursor [Danio rerio]
gi|7542472|gb|AAF63469.1|AF227738_1[7542472]
20: AAF63468
mannose binding-like lectin precursor [Cyprinus carpio]
gi|7542470|gb|AAF63468.1|AF227737_1[7542470]
21: AAK97540
surfactant protein A precursor [Gallus gallus]
gi|15420996|gb|AAK97540.1|AF411083_1[15420996]
22: LNMSMC
mannose-binding lectin C precursor - mouse
gi|7428747|pir||LNMSMC[7428747]
23: LNMSMA
mannose-binding lectin A precursor - mouse
gi|625320|pir||LNMSMA[625320]
24: JN0450
conglutinin precursor - bovine
gi|346501|pir||JN0450[346501]
25: A57250
mannan-binding protein - chicken (fragment)
gi|1362725|pir||A57250[1362725]
26: A53570
collectin-43 - bovine
gi|1083017|pir||A53570[1083017]
27: AAF28384
lung surfactant protein A [Sus scrofa]
gi|6782434|gb|AAF28384.1|AF133668_1[6782434]
28: AAF22145
lung surfactant protein D precursor; SPD; SP-D; CP4 [Sus scrofa]
gi|6760482|gb|AAF22145.2|AF132496_1[6760482]
29: P41317
MANNOSE-BINDING PROTEIN C PRECURSOR (MBP-C) (MANNAN-BINDING
PROTEIN)
(RA-REACTIVE FACTOR P28A SUBUNIT) (RARF/P28A)
gi|1346477|sp|P41317|MABC_MOUSE[1346477]
30: P39039
MANNOSE-BINDING PROTEIN A PRECURSOR (MBP-A) (MANNAN-BINDING
PROTEIN)
(RA-REACTIVE FACTOR POLYSACCHARIDE-BINDING COMPONENT P28B
POLYPEPTIDE) (RARF
P28B)
gi|729972|sp|P39039|MABA_MOUSE[729972]
31: P42916
COLLECTIN-43 (CL-43)
gi|1168967|sp|P42916|CL43_BOVIN[1168967]
32: CAB56155
DMBT1/8kb.2 protein [Homo sapiens]
gi|5912464|emb|CAB56155.1|[5912464]
33: BAA81747
collectin 34 [Homo sapiens]
gi|5162875|dbj|BAA81747.1|[5162875]
34: AAB94071
mannan-binding lectin; collectin [Gallus gallus]
gi|2736145|gb|AAB94071.1|[2736145]
35: AAB36019
mannan-binding protein, MBP = lectin {N-terminal} [chickens, serum, Peptide
Partial, 30 aa] [Gallus gallus]
gi|1311692|gb|AAB36019.1|[1311692]
36: AAB27504
conglutinin (N) {N-terminal} [cattle, Peptide Partial, 60 aa] [Bos taurus]
gi|386660|gb|AAB27504.1|[386660]
37: CAA53511
collectin-43 [Bos taurus]
gi|499385|emb|CAA53511.1|[499385]
38: AAA82010
mannose-binding protein C [Mus musculus]
gi|773288|gb|AAA82010.1|[773288]
39: AAA82009
mannose-binding protein A [Mus musculus]
gi|773280|gb|AAA82009.1|[773280]

Lung Surfactant Protein

1: 1KMRA
Chain A, Solution Nmr Structure Of Surfactant Protein B (11-25) (Sp-B11-25)
gi|22219056|pdb|1KMR|A[22219056]
2: P50404
Pulmonary surfactant-associated protein D precursor (SP-D) (PSP-D)
gi|1709879|sp|P50404|PSPD_MOUSE[1709879]
3: P06908
Pulmonary surfactant-associated protein A precursor (SP-A) (PSP-A) (PSAP)
gi|1172693|sp|P06908|PSPA_CANFA[1172693]
4: P35247
Pulmonary surfactant-associated protein D precursor (SP-D) (PSP-D)
gi|464486|sp|P35247|PSPD_HUMAN[464486]
5: P12842
Pulmonary surfactant-associated protein A precursor (SP-A) (PSP-A) (PSAP)
gi|131413|sp|P12842|PSPA_RABIT[131413]
6: NP_033186
surfactant associated protein D [Mus musculus]
gi|6677921|ref|NP_033186.1|[6677921]
7: 1B08C
Chain C, Lung Surfactant Protein D (Sp-D) (Fragment)
gi|6573321|pdb|1B08|C[6573321]
8: 1B08B
Chain B, Lung Surfactant Protein D (Sp-D) (Fragment)
gi|6573320|pdb|1B08|B[6573320]
9: 1B08A
Chain A, Lung Surfactant Protein D (Sp-D) (Fragment)
gi|6573319|pdb|1B08|A[6573319]
10: NP_060049
deleted in malignant brain tumors 1 isoform c precursor [Homo sapiens]
gi|8923740|ref|NP_060049.1|[8923740]
11: NP_015568
deleted in malignant brain tumors 1 isoform b precursor [Homo sapiens]
gi|6633801|ref|NP_015568.1|[6633801]
12: NP_004397
deleted in malignant brain tumors 1 isoform a precursor [Homo sapiens]
gi|4758170|ref|NP_004397.1|[4758170]
13: LNBOC1
pulmonary surfactant protein C - bovine
gi|7428752|pir||LNBOC1[7428752]
14: LNDGC1
pulmonary surfactant protein C - dog
gi|7428750|pir||LNDGC1[7428750]
15: JN0450
conglutinin precursor - bovine
gi|346501|pir||JN0450[346501]
16: A45225
pulmonary surfactant protein D precursor - human
gi|346375|pir||A45225[346375]
17: LNHUC
pulmonary surfactant protein C precursor, long splice form - human
gi|71983|pir||LNHUC[71983]
18: LNDGPS
pulmonary surfactant protein A precursor - dog
gi|71970|pir||LNDGPS[71970]
19: LNHUPS
pulmonary surfactant protein A precursor (genomic clone) - human
gi|71967|pir||LNHUPS[71967]
20: A53570
collectin-43 - bovine
gi|1083017|pir||A53570[1083017]
21: S33603
surfactant protein D - bovine
gi|423283|pir||S33603[423283]
22: AAF28384
lung surfactant protein A [Sus scrofa]
gi|6782434|gb|AAF28384.1|AF133668_1[6782434]
23: AAF22145
lung surfactant protein D precursor; SPD; SP-D; CP4 [Sus scrofa]
gi|6760482|gb|AAF22145.2|AF132496_1[6760482]
24: P15783
PULMONARY SURFACTANT-ASSOCIATED PROTEIN C (SP-C) (PULMONARY
SURFACTANT-ASSOCIATED PROTEOLIPID SPL(VAL))
gi|131422|sp|P15783|PSPC_BOVIN[131422]
25: P35246
PULMONARY SURFACTANT-ASSOCIATED PROTEIN D PRECURSOR (SP-D)
(PSP-D)
gi|464485|sp|P35246|PSPD_BOVIN[464485]
26: P42916
COLLECTIN-43 (CL-43)
gi|1168967|sp|P42916|CL43_BOVIN[1168967]
27: CAB56155
DMBT1/8kb.2 protein [Homo sapiens]
gi|5912464|emb|CAB56155.1|[5912464]
28: AAD49696
gp-340 variant protein [Homo sapiens]
gi|5733598|gb|AAD49696.1|AF159456_1[5733598]
29: AAD31380
surfactant protein D precursor [Mus musculus]
gi|4877556|gb|AAD31380.1|AF047742_1[4877556]
30: B61249
pulmonary surfactant protein C - dog
gi|539712|pir||B61249[539712]
31: S00609
pulmonary surfactant protein C - bovine
gi|89749|pir||S00609[89749]
32: A43628
pulmonary surfactant protein A - human (fragments)
gi|280854|pir||A43628[280854]
33: AAB48076
Surfactant protein B (SP-B) [Oryctolagus cuniculus]
gi|1850933|gb|AAB48076.1|[1850933]
34: 1901176A
surfactant protein A
gi|382753|prf||1901176A[382753]
35: CAA53510
lung surfactant protein D [Bos taurus]
gi|415939|emb|CAA53510.1|[415939]
36: CAA53511
collectin-43 [Bos taurus]
gi|499385|emb|CAA53511.1|[499385]
37: CAA46152
lung surfactant protein D [Homo sapiens]
gi|34767|emb|CAA46152.1|[34767]
38: AAA92788
lung surfactant protein C [Rattus norvegicus]
gi|595282|gb|AAA92788.1|[595282]
39: AAA31468
surfactant protein A [Oryctolagus cuniculus]
gi|431446|gb|AAA31468.1|[431446]

Mannose Binding Lectin

1: Q9NPY3
Complement component C1q receptor precursor (Complement component 1, q
subcomponent, receptor 1) (C1qRp) (C1qR(p)) (C1q/MBL/SPA receptor) (CD93
antigen) (CDw93)
gi|21759074|sp|Q9NPY3|CD93_HUMAN[21759074]
2: O89103
Complement component C1q receptor precursor (Complement component 1, q
subcomponent, receptor 1) (C1qRp) (C1qR(p)) (C1q/MBL/SPA receptor) (CD93
antigen) (Cell surface antigen AA4) (Lymphocyte antigen 68)
gi|21541998|sp|O89103|CD93_MOUSE[21541998]
3: P09871
Complement C1s component precursor (C1 esterase)
gi|115205|sp|P09871|C1S_HUMAN[115205]
4: NP_036204
complement component 1, q subcomponent, receptor 1; complement component
C1q
receptor [Homo sapiens]
gi|6912282|ref|NP_036204.1|[6912282]
5: NP_000233
soluble mannose-binding lectin precursor; mannose-binding lectin; mannose
binding protein; Mannose-binding lectin 2, soluble (opsonic defect) [Homo
sapiens]
gi|4557739|ref|NP_000233.1|[4557739]
6: AAM94381
lectin precursor [Zephyranthes candida]
gi|22212748|gb|AAM94381.1|AF527385_1[22212748]
7: AAH21762
mannose binding lectin, liver (A) [Mus musculus]
gi|18256010|gb|AAH21762.1|[18256010]
8: AAH10760
Similar to mannose binding lectin, serum (C) [Mus musculus]
gi|14789670|gb|AAH10760.1|[14789670]
9: P11226
Mannose-binding protein C precursor (MBP-C) (MBP1) (Mannan-binding protein)
(Mannose-binding lectin)
gi|126676|sp|P11226|MABC_HUMAN[126676]
10: NP_034897
mannan-binding lectin serine protease 2 [Mus musculus]
gi|6754642|ref|NP_034897.1|[6754642]
11: Q9ET61
Complement component C1q receptor precursor (Complement component 1, q
subcomponent, receptor 1) (C1qRp) (C1qR(p)) (C1q/MBL/SPA receptor) (CD93
antigen) (Cell surface antigen AA4)
gi|21541989|sp|Q9ET61|CD93_RAT[21541989]
12: NP_006601
mannan-binding lectin serine protease 2, isoform 1 precursor; MBL-associated
plasma protein of 19 kD; small MBL-associated protein [Homo sapiens]
gi|21264363|ref|NP_006601.2|[21264363]
13: NP_631947
mannan-binding lectin serine protease 2, isoform 2 precursor; MBL-associated
plasma protein of 19 kD; small MBL-associated protein [Homo sapiens]
gi|21264361|ref|NP_631947.1|[21264361]
14: NP_624302
mannan-binding lectin serine protease 1, isoform 2, precursor; protease, serine,
5 (mannose-binding protein-associated); manan-binding lectin serine protease-1;
Ra-reactive factor serine protease p100 [Homo sapiens]
gi|21264359|ref|NP_624302.1|[21264359]
15: NP_001870
mannan-binding lectin serine protease 1, isoform 1, precursor; protease, serine,
5 (mannose-binding protein-associated); manan-binding lectin serine protease-1;
Ra-reactive factor serine protease p100 [Homo sapiens]
gi|21264357|ref|NP_001870.3|[21264357]
16: XP_122683
similar to mannose binding lectin, liver (A) [Mus musculus]
gi|20872845|ref|XP_122683.1|[20872845]
17: AAM21196
C-type mannose-binding lectin [Oncorhynchus mykiss]
gi|20385163|gb|AAM21196.1|AF363271_1[20385163]
18: AAD45377
mannose-binding lectin [Sus scrofa]
gi|5566370|gb|AAD45377.1|AF164576_1[5566370]
19: NP_034905
mannose binding lectin, liver (A) [Mus musculus]
gi|6754654|ref|NP_034905.1|[6754654]
20: NP_034906
mannose binding lectin, serum (C) [Mus musculus]
gi|6754656|ref|NP_034906.1|[6754656]
21: AAL14428
dendritic cell-specific ICAM-3 grabbing nonintegrin [Macaca nemestrina]
gi|16118455|gb|AAL14428.1|AF343727_1[16118455]
22: AAF63470
mannose binding-like lectin precursor [Carassius auratus]
gi|7542474|gb|AAF63470.1|AF227739_1[7542474]
23: AAF63469
mannose binding-like lectin precursor [Danio rerio]
gi|7542472|gb|AAF63469.1|AF227738_1[7542472]
24: AAF63468
mannose binding-like lectin precursor [Cyprinus carpio]
gi|7542470|gb|AAF63468.1|AF227737_1[7542470]
25: AAF21018
mannose-binding lectin 2 [Sus scrofa]
gi|6644342|gb|AAF21018.1|AF208528_1[6644342]
26: AAK30298
mannose-binding lectin precursor protein [Gallus gallus]
gi|13561409|gb|AAK30298.1|[13561409]
27: LNMSMC
mannose-binding lectin C precursor - mouse
gi|7428747|pir||LNMSMC[7428747]
28: LNMSMA
mannose-binding lectin A precursor - mouse
gi|625320|pir||LNMSMA[625320]
29: LNRTMA
mannose-binding lectin A precursor - rat
gi|71975|pir||LNRTMA[71975]
30: LNRTMC
mannose-binding lectin C precursor - rat
gi|71974|pir||LNRTMC[71974]
31: LNHUMC
mannose-binding lectin precursor - human
gi|71973|pir||LNHUMC[71973]
32: BAA86864
complement C1s [Homo sapiens]
gi|6407558|dbj|BAA86864.1|[6407558]
33: P49329
MANNOSE-SPECIFIC LECTIN (AGGLUTININ)
gi|1346426|sp|P49329|LEC_ALOAR[1346426]
34: CAB56124
mannose-binding lectin [Homo sapiens]
gi|5911809|emb|CAB56124.1|[5911809]
35: CAB56123
mannose-binding lectin [Homo sapiens]
gi|5911807|emb|CAB56123.1|[5911807]
36: CAB56122
mannose-binding lectin [Homo sapiens]
gi|5911798|emb|CAB56122.1|[5911798]
37: CAB56121
mannose-binding lectin [Homo sapiens]
gi|5911796|emb|CAB56121.1|[5911796]
38: CAB56045
mannose-binding lectin [Homo sapiens]
gi|5911794|emb|CAB56045.1|[5911794]
39: CAB56120
mannose-binding lectin [Homo sapiens]
gi|5911792|emb|CAB56120.1|[5911792]
40: CAB56044
mannose-binding lectin [Homo sapiens]
gi|5911790|emb|CAB56044.1|[5911790]
41: AAB53110
C1qR(p) [Homo sapiens]
gi|2052498|gb|AAB53110.1|[2052498]

The collectin preferably comprises at least 10, such as at least 12, for example at least 15, such as at least 20, for example at least 25, such as at least 30, for example at least 35, such as at least 40, for example at least 50 consecutive amino acid residues of the collectin or of a variant or a homologue to said collectin. Such a variant or homologue is preferably at least 70%, such as 80%, for example 90%, such as 95% identical to the collectin.

Ficolins

The ficolin according to the invention may be L-ficolin, H-ficolin or M-ficolin or variants or homologues thereof. In a preferred embodiment the ficolin is L-ficolin.

In a particular preferred embodiment the ficolin has one of the sequences listed below with reference to their database and accession No. For each of the sequences the Cystein rich region and the collagen-like region is described.

```
NP_003656. ficolin 3 precursor; ficolin (collagen/fibrinogen domain-containing) 3
(Hakata antigen) [Homo sapiens] [gi: 4504331]
90 . . . 299/region_name = “pfam00147, fibrinogen_C, Fibrinogen beta and gamma
chains, C-terminal globular domain”
90 . . . 299/region_name = “smart00186, FBG, Fibrinogen-related domains (FReDs);
Domain present at the C-termini of fibrinogen beta and gamma chains, and a variety
of fibrinogen-related proteins, including tenascin and Drosophila scabrous”
  1 mdllwilpsl wllllggpac lktqehpscp gpreleaskv vllpscpgap gspgekgapg
  61 pqgppgppgk mgpkgepgdp vnllrcqegp rncrellsqg atlsgwyhlc lpegralpvf
121 cdmdtegggw lvfqrrqdgs vdffrswssy ragfgnqese fwlgnenlhq ltlqgnwelr
181 veledfngnr tfahyatfrl lgevdhyqla lgkfsegtag dslslhsgrp fttydadhds
241 snsncavivh gawwyascyr snlngryavs daaahkygid wasgrgvghp yrrvrmmlr
XP_116792. similar to Ficolin 2 precursor (Collagen/fibrinogen domain-containing
protein 2) (Ficolin-B) (Ficolin B) (Serum lectin P35) (EBP-37) (Hucolin) (L-Ficolin)
[Homo sapiens] [gi: 20477458]
91 . . . 168/region_name = “pfam00147, fibrinogen_C, Fibrinogen beta and gamma
chains, C-terminal globular domain”
91 . . . 168/region_name = “smart00186, FBG, Fibrinogen-related domains (FReDs);
Domain present at the C-termini of fibrinogen beta and gamma chains, and a variety
of fibrinogen-related proteins, including tenascin and Drosophila scabrous”
  1 mgpallalsf lwtmaltedt cpamleyval nsepgmaskn psrrhglsll vvdqgpgarg
  61 vrtdqgpsga dpgslelhge cpifpseqvi lthhnnypfs tedqdndrda encavhyqga
121 wwyaschlsh lngvylggar dsftnginwk sgkgnnysyk vsemkvrpt
O00602. Ficolin 1 precursor (Collagen/fibrinogen domain-containing protein 1)
(Ficolin-A) (Ficolin A) (M-Ficolin) [gi: 20455484]
1 . . . 29/gene = “FCN1”/region_name = “Signal”/note = “POTENTIAL.”
30 . . . 326/gene = “FCN1”/region_name = “Mature chain”/note = “FICOLIN 1.”
55 . . . 93/gene = “FCN1”/region_name = “Domain”/note = “COLLAGEN-LIKE.”
133/gene = “FCN1”/region_name = “Conflict”/note = “T −> N (IN REF. 1).”
144 . . . 290/gene = “FCN1”/region_name = “Domain”/note = “FIBRINOGEN
C-TERMINAL.”
287/gene = “FCN1”/region_name = “Conflict”/note = “N −> S (IN REF. 1).”
305/gene = “FCN1”/site_type = “glycosylation”/note = “N-LINKED (GLCNAC . . . )
(POTENTIAL).“
  1 melsgatmar glavllvlfl hiknlpaqaa dtcpevkvvg legsdkltil rgcpglpgap
  61 gpkgeagvig ergerglpga pgkagpvgpk gdrgekgmrg ekgdagqsqs catgprnckd
121 lldrgyflsg whtiylpdcr pltvlcdmdt dgggwtvfqr rmdgsvdfyr dwaaykqgfg
181 sqlgefwlgn dnihaltaqg sselrvdlvd fegnhqfaky ksfkvadeae kyklvlgafv
241 ggsagnsltg hnnnffstkd qdndvsssnc aekfqgawwy adchasnlng lylmgphesy
301 anginwsaak gykysykvse mkvrpa//
O75636. Ficolin 3 precursor (Collagen/fibrinogen domain-containing protein 3)
(Collagen/fibrinogen domain-containing lectin 3 P35) (Hakata antigen) [gi: 13124185]
1 . . . 21/gene = “FCN3”/region_name = “Signal”/note = “POTENTIAL.”
22 . . . 299/gene = “FCN3”/region_name = “Mature chain”/note = “FICOLIN 3.”
48 . . . 80/gene = “FCN3”/region_name = “Domain”/note = “COLLAGEN-LIKE.”
50/gene = “FCN3”/site_type = “hydroxylation”
53/gene = “FCN3”/site_type = “hydroxylation”
59/gene = “FCN3”/site_type = “hydroxylation”
65/gene = “FCN3”/site_type = “hydroxylation”
68/gene = “FCN3”/site_type = “hydroxylation”
77/gene = “FCN3”/site_type = “hydroxylation”
119 . . . 265/gene = “FCN3”/region_name = “Domain”/note = “FIBRINOGEN
C-TERMINAL.”
189/gene = “FCN3”/site_type = “glycosylation”/note = “N-LINKED (GLCNAC . . . )
(POTENTIAL).”
  1 mdllwilpsl wllllggpac lktqehpscp gpreleaskv vllpscpgap gspgekgapg
  61 pqgppgppgk mgpkgepgdp vnllrcqegp rncrellsqg atlsgwyhlc lpegralpvf
121 cdmdtegggw lvfqrrqdgs vdffrswssy ragfgnqese fwlgnenlhq ltlqgnwelr
181 veledfngnr tfahyatfrl lgevdhyqla lgkfsegtag dslslhsgrp fttydadhds
241 snsncavivh gawwyascyr snlngryavs daaahkygid wasgrgvghp yrrvrmmlr
XP_130120. similar to Ficolin 2 precursor (Collagen/fibrinogen domain-containing
protein 2) (Ficolin-B) (Ficolin B) (Serum lectin P35) (EBP-37) (Hucolin) [Mus mus-
culus] [gi: 20823464]
59 . . . 95/region_name = “Collagen triple helix repeat (20 copies)”/note = “Collagen”/
db_xref = “CDD: pfam01391”
59 . . . 89/region_name = “Collagen triple helix repeat (20 copies)”/note = “Collagen”/
db_xref = “CDD: pfam01391”
60 . . . 95/region_name = “Collagen triple helix repeat (20 copies)”/note = “Collagen”/
db_xref = “CDD: pfam01391”
60 . . . 95/region_name = “Collagen triple helix repeat (20 copies)”/note = “Collagen”/
db_xref = “CDD: pfam01391”
60 . . . 95/region_name = “Collagen triple helix repeat (20 copies)”/note = “Collagen”/
db_xref = “CDD: pfam01391”
60 . . . 95/region_name = “Collagen triple helix repeat (20 copies)”/note = “Collagen”/
db_xref = “CDD: pfam01391”
60 . . . 95/region_name = “Collagen triple helix repeat (20 copies)”/note = “Collagen”/
db_xref = “CDD: pfam01391”
61 . . . 95/region name = “Collagen triple helix repeat (20 copies)”/note = “Collagen”/
db_xref = “CDD: pfam01391”
61 . . . 95/region name = “Collagen triple helix repeat (20 copies)”/note = “Collagen”/
db_xref = “CDD: pfam01391”
61 . . . 95/region_name = “Collagen triple helix repeat (20 copies)”/note = “Collagen”/
db_xref = “CDD: pfam01391”
103 . . . 312/region_name = “Fibrinogen beta and gamma chains, C-terminal globular
domain”/note = “fibrinogen_C”/db_xref = “CDD: pfam00147”
103 . . . 312/region_name = “Fibrinogen-related domains (FReDs)”/note = “FBG”/
db_xref = “CDD: smart00186”
  1 malgsaalfv ltltvhaagt cpelkvldle gykqltilqg cpglpgaagp kgeagakgdr
  61 gesglpgipg kegptgpkgn qgekgirgek gdsgpsqsca tgprtckell tqghfltgwy
121 tiylpdcrpl tvlcdmdtdg ggwtvfqrrl dgsvdffrdw tsykrgfgsq lgefwlgndn
181 ihalttqgts elrvdlsdfe gkhdfakyss fqiqgeaeky klilgnflgg gagdsltphn
241 nrlfstkdqd ndgstsscam gyhgawwysq chtsnlngly lrgphksyan gvnwkswrgy
301 nysckvsemk vrli
NP_056654. ficolin 2 isoform d precursor; ficolin (collagen/fibrinogen domain-
containing lectin) 2 (hucolin); ficolin (collagen/fibrinogen domain-containing lectin) 2;
hucolin [Homo sapiens] [gi: 8051590]
39 . . . 95/region_name = “collagen-like domain”
  1 meldravgvl gaatlllsfl gmawalqaad tcpevkmvgl egsdkltilr gcpglpgapg
  61 dkgeagtngk rgergppgpp gkagppgpng apgepqpclt gd
NP_056653. ficolin 2 isoform c precursor; ficolin (collagen/fibrinogen domain-
containing lectin) 2 (hucolin); ficolin (collagen/fibrinogen domain-containing lectin) 2;
hucolin [Homo sapiens] [gi: 8051588]
39 . . . 95/region_name = “collagen-like domain”
102 . . . 143/region_name = “Fibrinogen beta and gamma chains, C-terminal globular
domain”/note = “fibrinogen_C”/db_xref = “CDD: pfam00147”
102 . . . 143/region_name = “Fibrinogen-related domains (FReDs)”/note = “FBG”/
db_xref = “CDD: smart00186”
  1 meldravgvl gaatlllsfl gmawalqaad tcpevkmvgl egsdkltilr gcpglpgapg
  61 dkgeagtngk rgergppgpp gkagppgpng apgepqpclt gprtckdlld rghflsgwht
121 iylpdcrplt vlcdmdtdgg gwtvsvglgr ggqpgspggq aahlvgehtl efsillvgds
181 qr
NP_056652. ficolin 2 isoform b precursor; ficolin (collagen/fibrinogen domain-
containing lectin) 2 (hucolin); ficolin (collagen/fibrinogen domain-containing lectin) 2;
hucolin [Homo sapiens] [gi: 8051586]
sig_peptide 1 . . . 25
mat_peptide 26 . . . 275
60 . . . 275/region_name = “FBG domain”/note = “fibrinogen beta/gamma homology”
64 . . . 275/region_name = “Fibrinogen-related domains (FReDs)”/note = “FBG”/
db_xref = “CDD: smart00186”
64 . . . 274/region_name = “Fibrinogen beta and gamma chains, C-terminal globular
domain”/note = “fibrinogen_C”/db_xref = “CDD: pfam00147”
  1 meldravgvl gaatlllsfl gmawalqaad tcpgergppg ppgkagppgp ngapgepqpc
  61 ltgprtckdl ldrghflsgw htiylpdcrp ltvlcdmdtd gggwtvfqrr vdgsvdfyrd
121 watykqgfgs rlgefwlgnd nihaltaqgt selrvdlvdf ednyqfakyr sfkvadeaek
181 ynlvlgafve gsagdsltfh nnqsfstkdq dndlntgnca vmfqgawwyk nchvsnlngr
241 ylrgthgsfa nginwksgkg ynysykvsem kvrpa
NP_001994. ficolin 1 precursor; ficolin (collagen/fibrinogen domain-containing) 1
[Homo sapiens] [gi: 8051584]
sig_peptide 1 . . . 27
mat_peptide 28 . . . 326
40 . . . 108/region_name = “collagen-like domain”
50 . . . 105/region_name = “Collagen triple helix repeat (20 copies)”/note = “Collagen”/
db_xref = “CDD: pfam01391”
51 . . . 107/region name = “Collagen triple helix repeat (20 copies)”/note = “Collagen”/
db_xref = “CDD: pfam01391”
52 . . . 106/region_name = “Collagen triple helix repeat (20 copies)”/note = “Collagen”/
db_xref = “CDD: pfam01391”
115 . . . 326/region_name = “FBG domain”/note = “fibrinogen beta/gamma homology”
115 . . . 326/region_name = “Fibrinogen-related domains (FReDs)”/note = “FBG”/
db_xref = “CDD: smart00186”
115 . . . 325/region_name = “Fibrinogen beta and gamma chains, C-terminal globular
domain”/note = “fibrinogen C”/db_xref = “CDD: pfam00147” variation 315/
db_xref = “dbSNP: 1128428” variation 316/db_xref = “dbSNP: 1128429” variation 317/
db_xref = “dbSNP: 1128430”
  1 melsgatmar glavllvlfl hiknlpaqaa dtcpevkvvg legsdkltil rgcpglpgap
  61 gpkgeagvig ergerglpga pgkagpvgpk gdrgekgmrg ekgdagqsqs catgprnckd
121 lldrgyflsg whtiylpdcr pltvlcdmdt dgggwtvfqr rmdgsvdfyr dwaaykqgfg
181 sqlgefwlgn dnihaltaqg sselrvdlvd fegnhqfaky ksfkvadeae kyklvlgafv
241 ggsagnsltg hnnnffstkd qdndvsssnc aekfqgawwy adchasnlng lylmgphesy
301 anginwsaak gykysykvse mkvrpa
NP_004099. ficolin 2 isoform a precursor; ficolin (collagen/fibrinogen domain-
containing lectin) 2 (hucolin); ficolin (collagen/fibrinogen domain-containing lectin) 2;
hucolin [Homo sapiens] [gi: 4758348]
sig_peptide 1 . . . 25
mat_peptide 26 . . . 313
39 . . . 95/region_name = “collagen-like domain”
98 . . . 313/region_name = “FBG domain”/note = “fibrinogen beta/gamma homology”
102 . . . 313/region_name = “Fibrinogen-related domains (FReDs)”/note = “FBG”/
db_xref = “CDD: smart00186”
102 . . . 312/region_name = “Fibrinogen beta and gamma chains, C-terminal globular
domain”/note = “fibrinogen_C”/db_xref = “CDD: pfam00147
  1 meldravgvl gaatlllsfl gmawalqaad tcpevkmvgl egsdkltilr gcpglpgapg
  61 dkgeagtngk rgergppgpp gkagppgpng apgepqpclt gprtckdlld rghflsgwht
121 iylpdcrplt vlcdmdtdgg gwtvfqrrvd gsvdfyrdwa tykqgfgsrl gefwlgndni
181 haltaqgtse lrvdlvdfed nyqfakyrsf kvadeaekyn lvlgafvegs agdsltfhnn
241 qsfstkdqdn dlntgncavm fqgawwyknc hvsnlngryl rgthgsfang inwksgkgyn
301 ysykvsemkv rpa
Q9WTS8. Ficolin 1 precursor (Collagen/fibrinogen domain-containing protein 1)
(Ficolin-A) (Ficolin A) (M-Ficolin) [gi: 13124116]
1 . . . 22/gene = “FCN1”/region_name = “Signal”/note = “POTENTIAL.”
23 . . . 335/gene=“FCN1”/region_name = “Mature chain”/note = “FICOLIN 1.”
50 . . . 88/gene = “FCN1”/region_name = “Domain”/note = “COLLAGEN-LIKE.”
152 . . . 298/gene = “FCN1”/region_name = “Domain”/note = “FIBRINOGEN
C-TERMINAL.”
271/gene = “FCN1”/site_type = “glycosylation”/note = “N-LINKED (GLCNAC . . . )
(POTENTIAL).”
  1 mwwpmlwafp vllclcssqa lgqesgacpd vkivglgaqd kvaviqscps fpgppgpkge
  61 pgspagrger glqgspgkmg ppgskgepgt mgppgvkgek gergtasplg qkelgdalcr
121 rgprsckdll trgifltgwy tiylpdcrpl tvlcdmdvdg ggwtvfqrrv dgsinfyrdw
181 dsykrgfgnl gtefwlgndy lhlltangnq elrvdlrefq gqtsfakyss fqvsgeqeky
241 kltlgqfleg tagdsltkhn nmafsthdqd ndtnggknca alfhgawwyh dchqsnlngr
301 ylpgshesya dginwlsgrg hrysykvaem kiras
Q15485. Ficolin 2 precursor (Collagen/fibrinogen domain-containing protein 2)
(Ficolin-B) (Ficolin B) (Serum lectin P35) (EBP-37) (Hucolin) (L-Ficolin) [gi: 13124203]
1 . . . 25/gene = “FCN2”/region_name = “Signal”/note = “POTENTIAL.”
26 . . . 313/gene = “FCN2”/region_name = “Mature chain”/note = “FICOLIN 2.”
54 . . . 92/gene = “FCN2”/region_name = “Domain”/note = “COLLAGEN-LIKE.”
131 . . . 277/gene = “FCN2”/region_name = “Domain”/note = “FIBRINOGEN
C-TERMINAL”
240/gene = “FCN2”/site_type = “glycosylation”/note = “N-LINKED (GLCNAC . . . )
(POTENTIAL).”
300/gene = “FCN2”/site_type = “glycosylation”/note = “N-LINKED (GLCNAC . . . )
(POTENTIAL).”
  1 meldravgvl gaatlllsfl gmawalqaad tcpevkmvgl egsdkltilr gcpglpgapg
  61 dkgeagtngk rgergppgpp gkagppgpng apgepqpclt gprtckdlld rghflsgwht
121 iylpdcrplt vlcdmdtdgg gwtvfqrrvd gsvdfyrdwa tykqgfgsrl gefwlgndni
181 haltaqgtse lrvdlvdfed nyqfakyrsf kvadeaekyn lvlgafvegs agdsltfhnn
241 qsfstkdqdn dlntgncavm fqgawwyknc hvsnlngryl rgthgsfang inwksgkgyn
301 ysykvsemkv rpa
O70497. Ficolin 2 precursor (Collagen/fibrinogen domain-containing protein 2)
(Ficolin-B) (Ficolin B) (Serum lectin P35) (EBP-37) (Hucolin) [gi: 13124181]
<1 . . . 15/gene = “FCN2”/region_name = “Signal”/note = “POTENTIAL.”
16 . . . >306/gene = “FCN2”/region_name = “Mature chain”/note = “FICOLIN 2.”
41 . . . 79/gene = “FCN2”/region_name = “Domain”/note = “COLLAGEN-LIKE.”
130 . . . 276/gene = “FCN2”/region_name = “Domain”/note = “FIBRINOGEN
C-TERMINAL.”
299/gene = “FCN2”/site_type = “glycosylation”/note = “N-LINKED (GLCNAC . . . )
(POTENTIAL).”
  1 lgsaalfvlt ltvhaagtcp elkvldlegy kqltilqgcp glpgaagpkg eagakgdrge
  61 sglpgipgke gptgpkgnqg ekgirgekgd sgpsqscatg prtckelltq ghfltgwyti
121 ylpdcrpmtv lcdmdtdggg wtvfqrrldg svdffrdwts ykrgfgsqlg efwlgndnih
181 alttqgtsel rvdlsdfegk hdfakyssfq iqgeaekykl ilgnflggga gdsltphnnr
241 lfstkdqdnd gstsscamgy hgawwysqch tsnlnglylr gphksyangv nwkswrgyny
301 sckvse
O70165. Ficolin 1 precursor (Collagen/fibrinogen domain-containing protein 1)
(Ficolin-A) (Ficolin A) (M-Ficolin) [gi: 13124179]
1 . . . 22/gene = “FCN1”/region_name = “Signal”/note = “POTENTIAL.”
23 . . . 334/gene = “FCN1”/region_name = “Mature chain”/note = “FICOLIN 1.”
50 . . . 88/gene = “FCN1”/region_name = “Domain”/note = “COLLAGEN-LIKE.”
152 . . . 298/gene = “FCN1”/region_name = “Domain”/note = “FIBRINOGEN
C-TERMINAL”
261/gene = “FCN1”/site_type = “glycosylation”/note = “N-LINKED (GLCNAC . . . )
(POTENTIAL).”
  1 mqwptlwafs gllclcpsqa lgqergacpd vkvvglgaqd kvvviqscpg fpgppgpkge
  61 pgspagrger gfqgspgkmg pagskgepgt mgppgvkgek gdtgaapslg ekelgdtlcq
121 rgprsckdll trgifltgwy tihlpdcrpl tvlcdmdvdg ggwtvfqrrv dgsidffrdw
181 dsykrgfgnl gtefwlgndy lhlltangnq elrvdlqdfq gkgsyakyss fqvseeqeky
241 kltlgqfleg tagdsltkhn nmsftthdqd ndansmncaa lfhgawwyhn chqsnlngry
301 lsgshesyad ginwgtgqgh hysykvaemk iras
P57756. Ficolin 2 precursor (Collagen/fibrinogen domain-containing protein 2)
(Ficolin-B) (Ficolin B) (Serum lectin P35) (EBP-37) (Hucolin) [gi: 13124114]
1 . . . 22/gene = “FCN2”/region_name = “Signal”/note = “POTENTIAL.”
23 . . . 319/gene = “FCN2”/region_name = “Mature chain”/note = “FICOLIN 2.”
48 . . . 86/gene = “FCN2”/region_name = “Domain”/note = “COLLAGEN-LIKE.”
137 . . . 283/gene = “FCN2”/region_name = “Domain”/note = “FIBRINOGEN
C-TERMINAL.”
306/gene = “FCN2”/site_type = “glycosylation”/note = “N-LINKED (GLCNAC . . . )
(POTENTIAL).”
  1 mvlgsaalfv lslcvteltl haadtcpevk vldlegsnkl tilqgcpglp galgpkgeag
  61 akgdrgesgl pghpgkagpt gpkgdrgekg vrgekgdtgp sqscatgprt ckelltrgyf
121 ltgwytiylp dcrpltvlcd mdtdgggwtv fqrridgtvd ffrdwtsykq gfgsqlgefw
181 lgndnihalt tqgtnelrvd ladfdgnhdf akyssfqiqg eaekyklilg nflgggagds
241 ltsqnnmlfs tkdqdndqgs sncavryhga wwysdchtsn lnglylrglh ksyangvnwk
301 swkgynysyk vsemkvrli
JC5980. ficolin-A precurs - mouse [gi: 7513652]
1 . . . 21/region_name = “domain”/note = “signal sequence”
50 . . . 64/region_name = “domain”/note = “collagen-like”
68 . . . 106/region_name = “domain”/note = “collagen-like”
123 . . . 334/region_name = “domain”/note = “fibrinogen beta/gamma homology #label
FBG”
  1 mqwptlwafs gllclcpsqa lgqergacpd vkvvglgaqd kvvviqscpg fpgppgpkge
  61 pgspagrger gfqgspgkmg pagskgepgt mgppgvkgek gdtgaapslg ekelgdtlcq
121 rgprsckdll trgifltgwy tihlpdcrpl tvlcdmdvdg ggwtvfqrrv dgsidffrdw
181 dsykrgfgnl gtefwlgndy lhlltangnq elrvdlqdfq gkgsyakyss fqvseeqeky
241 kltlgqfleg tagdsltkhn nmsftthdqd ndansmncaa lfhgawwyhn chqsnlngry
301 lsgshesyad ginwgtgqgh hysykvaemk iras
S61517. ficolin-1 precurs - human [gi: 2135116]
1 . . . 326/note = “36K HLA-cross-reactive plasma protein; hucolin, 35 K”
1 . . . 22/region_name = “domain”/note = “signal sequence”
52 . . . 108/region_name = “region”/note = “collagen-like”
115 . . . 326/region_name = “domain”/note = “fibrinogen beta/gamma homology #label
FBG”
305/site_type = “binding”/note = “carbohydrate (Asn) (covalent)”
  1 melsgatmar glavllvlfl hiknlpaqaa dtcpevkvvg legsdkltil rgcpglpgap
  61 gpkgeagvig ergerglpga pgkagpvgpk gdrgekgmrg ekgdagqsqs catgprnckd
121 lldrgyflsg whniylpdcr pltvlcdmdt dgggwtvfqr rmdgsvdfyr dwaaykqgfg
181 sqlgefwlgn dnihaltaqg sselrvdlvd fegnhqfaky ksfkvadeae kyklvlgafv
241 ggsagnsltg hnnnffstkd qdndvsssnc aekfqgawwy adchasslng lylmgphesy
301 anginwsaak gykysykvse mkvrpa
A47172. transforming growth factor-beta 1-binding protein homolog ficolin-alpha-
pig [gi: 423206]
112 . . . 323/region_name = “domain”/note = “fibrinogen beta/gamma homology #label
FBG”
  1 mdtrgvaaam rplvllvafl ctaapaldtc pevkvvgleg sdklsilrgc pglpgaagpk
  61 geagasgpkg gqgppgapge pgppgpkgdr gekgepgpkg esweteqclt gprtckellt
121 rghilsgwht iylpdcqplt vlcdmdtdgg gwtvfqrrsd gsvdfyrdwa aykrgfgsql
181 gefwlgndhi haltaqgtne lrvdlvdfeg nhqfakyrsf qvadeaekym lvlgafvegn
241 agdsltshnn slfttkdqdn dqyasncavl yqgawwynsc hvsnlngryl ggshgsfang
301 vnwssgkgyn ysykvsemkf rat
JC4942. ficolin-1 precursor - human [gi: 2135117]
1 . . . 22/region_name = “domain”/note = “signal sequence”
45 . . . 101/region_name = “region”/note = “collagen-like”
108 . . . 319/region_name = “domain”/note = “fibrinogen beta/gamma homology #label
FBG”
111 . . . 315/region_name = “region”/note = “fibrinogen-like”
298/site_type = “binding”/note = “carbohydrate (Asn) (covalent)”
  1 marglavllv lflhiknlpa qaadtcpevk vvglegsdkl tilrgcpglp gapgpkgeag
  61 vigergergl pgapgkagpv gpkgdrgekg mrgekgdagq sqscatgprn ckdlldrgyf
121 lsgwhtiylp dcrpltvlcd mdtdgggwtv fqrrmdgsvd fyrdwaaykq gfgsqlgefw
181 lgndnihalt aqgsselrvd lvdfegnhqf akyksfkvad eaekyklvlg afvggsagns
241 ltghnnnffs tkdqdndvss sncaekfqga wwyadchasn lnglylmgph esyanginws
301 aakgykysyk vsemkvrpa
AAF44911. symbol = BG: DS00929 . . . [gi: 7287873]
  1 mkscffvlfl wtllfevgqs sphtcpsgsp ngihqlmlpe eepfqvtqck ttardwiviq
  61 rrldgsvnfn qswfsykdgf gdpngeffig lqklylmtre qphelfiqlk hgpgatvyah
121 fddfqvdset elyklervgk ysgtagdslr yhinkrfstf drdndesskn caaehgggww
181 fhsclsr
```

The ficolin preferably comprises at least 10, such as at least 12, for example at least 15, such as at least 20, for example at least 25, such as at least 30, for example at least 35, such as at least 40, for example at least 50 consecutive amino acid residues of the ficolins identified above or of a variant or a homologue to said ficolin. Such a variant or homologue is preferably at least 70%, such as 80%, for example 90%, such as 95% identical to the complement activating protein.

In the following the invention is described in relation to MBL as an example:

SARS may be prevented when administering MBL to these individuals having an MBL level in excess of 10 ng/ml serum. Also, individuals having an MBL level in excess of 50 ng/ml serum may be in need of treatment, such as individuals having an MBL level in excess of 100 ng/ml serum, and individuals having an MBL level in excess of 150 ng/ml serum.

Also the MBL treatment of SARS may be conducted by administering MBL to these individuals in combination with relevant antibiotics, anti-viral agents or anti-fungal agents.

In particular, individuals at risk of acquiring SARS will benefit from being prophylactically treated with MBL.

Generally all individuals exposed to SARS patients should be treated with MBL independent on their specific MBL level. The reason behind this is that SARS may lead to MBL depletion, and therefore an MBL “booster”, increasing the MBL level initially will reduce the risk of MBL depletion to a level below a deficiency level, and the immune defence of these patients can be reinforced by administration of recombinant or natural plasma-derived MBL. In particular SARS may be prevented when administering MBL to individuals having an MBL level in excess of 10 ng/ml serum. Also, individuals having an MBL level in excess of 50 ng/ml serum may be in need of treatment, such as individuals having an MBL level in excess of 100 ng/ml serum, and individuals having an MBL level in excess of 150 ng/ml serum.

The present inventors have also shown herein that in particular individuals having an MBL level below 500 ng/ml serum will benefit from the MBL treatment. Consequently, in particular individuals having an MBL level below 400 ng/ml will benefit, such as individuals having an MBL level below 300 ng/ml, such as individuals having an MBL level below 250 ng/ml, such as individuals having an MBL level below 200 ng/ml.

Thus, in a preferred embodiment the present invention relates to the use of MBL for manufacturing of a medicament for treatment of individuals having an MBL level in serum in the range of 10-500 ng/ml, such as in the range of 50-500 ng/ml for treating and/or preventing SARS.

One group of individuals being in need of MBL treatment in order to prevent and/or treat SARS are individuals having a low level of functional MBL, independent on the level of MBL as such. This is due to the fact that for some mutations of the MBL it has been found that although MBL subunits and oligomers thereof are expressed in serum the functionality thereof are low. The functionality or functional activity of MBL may be estimated by its capacity to form an MBL/MASP complex leading to activation of the complement system. When C4 is cleaved by MBL/MASP an active thiolester is exposed and C4 becomes covalently attached to nearby nucleophilic groups. A substantial part of the C4b will thus become attached to the coated plastic well and may be detected by anti-C4 antibody.

A quantitative TRIFMA for MBL functional activity is constructed by 1) coating microtitre wells with 1 mg mannan in 100 ml buffer; 2) blocking with Tween-20; 3) applying test samples, e.g. diluted MBL preparations 4) applying MBL deficient serum (this leads to the formation of the MBL/MASP complex); alternatively the MBL and the MBL deficient serum may be mixed before application with the microtitre wells; 5) applying purified complement factor C4 at 5 mg/ml; 6) incubating for one hour at 37° C.; 7) applying Eu-labelled anti-C4 antibody; 8) applying enhancement solution; and 9) reading the Eu by time resolved fluorometry. Between each step the plate is incubated at room temperature and washed, except between step 8 and 9.

Estimation by ELISA may be carried out similarly, e.g. by applying biotin-labelled anti-C4 in step 7; 8) apply alkaline phosphatase-labelled avidin; 9) apply substrate; and 10) read the colour intensity.

The functionality may be expressed as the specific activity of MBL, such as 1 unit of MBL activity per ng MBL. A non-functional MBL may be defined as MBL having a specific activity less than 50% of plasma MBL specific activity, such as less than 25% of plasma MBL specific activity, wherein the plasma MBL is purified from an individual not suffering from any MBL mutations. In particular the reference plasma MBL is plasma pool LJ 6.57 Apr. 28, 1997.

Thus, the present invention also relates to the prevention and/or treatment of SARS in individuals having a mutation in their MBL gene leading to a reduced expression of MBL and/or expression of non-functional MBL.

In particular such mutations in the MBL gene can lead to a change of aminoacid number 52 (numbering including the leader peptide of MBL) from arginine to cysteine, aminoacid number 54 from glycine to aspartic acid or amino acid number 75 from glycine to glutamic acid.

Also mutations in the promoter region of the MBL gene can lead to lowered levels of MBL. In particular mutations at position −221 have an influence on the expression of MBL.

The MBL sequence may be found in swiss.prot under accession No: 11226.

The MBL composition used to manufacture an MBL medicament may be produced from any MBL source available. The MBL source may be natural MBL, whereby the MBLs are produced in a native host organism, meaning that MBL is produced by a cell normally expressing MBL. One usual method of producing an MBL composition is by extraction of MBL from human body liquids, such as serum or plasma, but MBL may also be harvested from cultures of hepatocytes.

In another aspect the MBL oligomers are produced by a host organism not natively expressing an MBL polypeptide, such as by recombinant technology.

In a first embodiment the MBL source may be serum, from which an MBL composition is obtained by purification from serum, plasma, milk product, colostrum or the like by a suitable purification method, such as affinity chromatography using carbohydrate-derivatised matrices, such as mannose or mannan coupled matrices. Such a method is discussed in WO99/64453, wherein the purification process is followed by a virus-removal step in order to remove infectious agents from the MBL source, since one of the major problems with proteins purified from body liquids is the risk of introducing infectious agents in combination with the desired protein. WO99/64453 is hereby incorporated by reference.

The MBL composition used to manufacture an MBL medicament preferably comprises MBL oligomers having a size distribution substantially identical to the size distribution of MBL in serum, such as a size distribution profile at least 50% identical to the size distribution profile of MBL in serum. By identical is meant that at least 50% of the oligomers has an apparent molecular weight higher than 200 kDa, when analysed by SDS-PAGE and/or Western blot.

In a more preferred embodiment the size distribution profile is at least 75% identical to the size distribution profile of MBL in serum, such as at least 90% identical to the size distribution profile of MBL in serum, and more preferred at least 95% identical to the size distribution profile of MBL in serum.

When purifying from an MBL source initially having another size distribution profile it is preferred that the affinity chromatography used to purify from the MBL source favours purification of oligomers having an apparent molecular weight higher than 200 kDa. This is obtained by using a carbohydrate-derivatized matrix having substantially no affinity to subunits and/or dimers of MBL. Preferably the carbohydrate-derivatized matrix has affinity for substantially only tetrameric, pentameric and/or hexameric recombinant MBLs.

The matrix may be derivatized with any carbohydrate or carbohydrate mixture whereto MBL binds and for which binding of the higher oligomers of MBL are favoured. The carbohydrate-derivatized matrix is preferably a hexose-derivatized matrix, such as a mannose- or a N-acetyl-glucosamin derivatized matrix, such as most preferably a mannose-derivatized matrix.

The selectivity of the carbohydrate-derivatized matrix is obtained by securing that the matrix as such, i.e the un-derivatized matrix has substantially no affinity to MBL polypeptides, in particular no affinity to MBL trimers or smaller oligomers. This may be ensured when the matrix as such is carbohydrate-free. In particular the matrix should not contain any Sepharose or the like. It is preferred that the matrix consists of a non-carbohydrate containing polymer material, such as Fractogel® TSK beads.

The matrix may be in any form suitable for the chromatography, mostly in the form of beads, such as plastic beads.

After application of the MBL source the column is washed, preferably by using non-denaturing buffers, having a composition, pH and ionic strength resulting in elimination of proteins, without eluting the higher oligomers of MBL. Such as buffer may be TBS. Elution of MBL is performed with a selective desorbing agent, capable of efficient elution of highed oligomers of MBL, such as TBS comprising a desorbing agent, such as EDTA (for example 5 mM EDTA) or mannose (for example 50 mM mannose), and MBL oligomers are collected. Such a purification method is described in co-pending International patent application No. WO 00/70043.

In a preferred aspect a clinical grade MBL composition is obtained by using an MBL source produced by recombinant technology, wherein the MBL source is the culture media from culturing of MBL producing cells.

Thus, the present invention encompasses MBL produced by a process of producing a recombinant mannan binding lectin (MBL), comprising the steps of:
preparing a gene expression construct comprising a DNA sequence encoding a MBL polypeptide or a functional equivalent thereof,
transforming a host cell culture with the construct,
cultivating the host cell culture, thereby obtaining expression and secretion of the polypeptide into the culture medium, followed by
obtaining a culture medium comprising human recombinant MBLs.

The culture medium comprising the human recombinant MBL polypeptides may then be processed as described above for purification of MBL.

The MBL polypeptide is preferably a mammalian MBL polypeptide, such as more preferably a human MBL polypeptide. The gene expression construct may be produced by conventional methods known to the skilled person, such as described in U.S. Pat. No. 5,270,199.

In another embodiment the gene expression construct is prepared as described in WO 00/70043.

The expression is preferably carried out in e.g. mammalian cells, the preparation according to the invention results from the use of an expression vector comprising intron sequence(s) from an MBL gene and at least one exon sequence. Regarding the transgenic animals as expression system this term is in this context animals which have been genetically modified to contain and express the human MBL gene or fragments or mimics hereof.

In addition to the purification method it is preferred that the gene expression construct and the host cell also favours production of higher oligomers, which has been found to be possible by using a gene expression construct comprising at least one intron sequence from the human MBL gene or a functional equivalent thereof. malian cells and cells from insects.

Consequently, the MBL composition may be used for preventing and/or treating SARS in an individual wherein the microbial species is a fungus, a yeast, a protozoa, a parasite and/or a bacteria.

The medicament may be produced by using the eluant obtained from the affinity chromatography as such. It is however preferred that the eluant is subjected to further purification steps before being used.

In addition to the MBL oligomers, the medicament may comprise a pharmaceutically acceptable carrier substance and/or vehicles. In particular, a stabilising agent may be added to stabilise the MBL proteins. The stabilising agent may be a sugar alcohol, saccharides, proteins and/or amino acids. Examples of stabilising agents may be maltose or albumin.

Other conventional additives may be added to the medicament depending on administration form for example. In one embodiment the medicament is in a form suitable for injections. Conventional carrier substances, such as isotonic saline, may be used.

In another embodiment the medicament is in a form suitable for pulmonal administration, such as in the form of a powder for inhalation or creme or fluid for topical application.

The route of administration may be any suitable route, such as intravenously, intramusculary, subcutanously or intradermally. Also, pulmonal or topical administration is envisaged by the present invention.

Normally from 1-100 mg is administered per dosage, such as from 2-10 mg, mostly from 5-10 mg per dosage depending on the individual to be treated, for example about 0.1 mg/kg body weight is administered.

The use of an MBL composition for the manufacture of a medicament may also further comprise the manufacture of another medicament, such as an anti-fungal, anti-yeast, anti-bacterial and/or anti-viral medicament for obtaining a kit-of-parts.

The anti-viral medicament may be a medicament capable of virus attenuation and/or elimination.

The invention also relates to an aspect of using a measurement of the MBL level as a prognostic marker for the risk of the individual of acquiring SARS and thereby an indicative of the need for treatment. In particular an MBL level below 500 ng/ml is a prognostic marker indicative for treatment with MBL.

Thus, the present invention also relates to a method of using an MBL composition for preventing and/or treating SARS in an individual, the method comprising the steps of:
i) determining serum levels of MBL in an individual,
ii) estimating the probability of the occurrence of a significant clinical SARS in the individual, and optionally,
   administering an MBL composition to the individual.

The MBL level is measured in serum or plasma, and may be determined by time resolved immunofluorescent assay (TRIFMA), ELISA, RIA or nephelometry.

Also the MBL levels may be inferred from analysis of genotypes of the MBL genes as discussed above in relation to mutations of MBL leading to a decreased MBL level.

The invention is illustrated in the following examples.

### EXAMPLE 1

MBL Serum Levels in Patients Suffering from SARS

Patients are selected among individuals presenting clinically significant SARS as defined above. Patients are identified by retrospective computer search of the patient database.

Before entering treatment blood is drawn into evacuated glass tubes containing EDTA (final concentration about 10 mM). The plasma is aliquoted and kept at −80° C. until assay. Plasma samples are similarly obtained from healthy blood donors. The patients are free of infections at the time of blood sampling.

The concentration of MBL is determined by a time resolved immunofluorescent assay (TRIFMA). Microtitre wells (fluoroNunc, Nunc, Kamstrup, Denmark) are coated with antibody by incubation overnight at room temperature with 500 ng anti-human MBL antibody (Mab 131-1, Statens Serum Institut, Copenhagen, Denmark) in 100 μl PBS (0.14 M NaCl, 10 mM phosphate, pH 7.4). After wash with Tween-containing buffer (TBS, 0.14 M NaCl, 10 mM Tris/HCl, 7.5 mM NaN₃, pH 7.4 with 0.05% Tween 20) test samples (plasma 1/20) and calibrator dilutions are added in TBS/Tween with extra NaCl to 0.5 M and 10 mM EDTA.

After overnight incubation at 4° C. and wash, the developing europium-labelled antibody (12.5 ng Mab 131-1 labelled with the Eu-containing chelate, isothiocyanato-benzoyl-diethylene-triamine-tetra acetic acid, according to the manufacturer, Wallac, Turku, Finland) is added in TBS/Tween with 25 μM EDTA.

Following incubation for 2 h and wash, fluorescence enhancement solution is added (Wallac) and the plates are read on a time resolved fluorometre (Delfia 1232, Wallac). The calibration curve is made using dilutions of one plasma, which is kept aliquoted at −80° C.

Based on the above outlined method, the MBL serum level of patients with SARS as compared to non-SARS patients is compared.

### EXAMPLE 2

Effect of MBL on SARS-Related Coronavirus (SARS-CoV) Infectivity

In the present Example, the following materials were used:
HBSS=Hanks balanced salt solution
PBS=phosphate buffered saline.
   Virus Preparation and Cells

Prototype SARS-CoV HKU39849 were grown on the fetal rhesus kidney cells (FRhK-4) (ATCC). The cells were grown in MEM medium with 10% fetal calf serum.

Virus titers (TCID₅₀) were determined by titration of a 10-fold dilution series on FRhK-4 cells.

Assessment of Binding of MBL to SARS-CoV Strains

Recombinat human MBL (Natimmune A/S) was used.

Binding of MBL to SARS-CoV strains was tested with an ELISA in which suspensions of virus (10³ to 10⁵ TCID₅₀) diluted in PBS were incubated overnight at 4 C on 96-well plates, followed by washing and incubation with MBL.

Before the addition of MBL, plates were blocked with BSA and gelatin in PBS and washed with HBSS+0.05% Tween 20.

MBL (0 to 10 μg/mL) diluted in Ca²⁺ containing HBSS were then added and allowed to incubate with virus for 2 hours, followed by the washing off of unbound lectin (three times in HBSS). Control experiments were done by addition of EDTA or mannan to some of the MBL containing wells.

The presence of bound MBL was detected with biotinylated monoclonal anti-MBL (HYB131-01, Antibody shop) diluted in HBSS/Ca²⁺. Biotin was detected with streptavidin conjugated to horseradish peroxidase and tetramethylbenzidine (‘TMB’) substrate. The reaction was stopped with 0.5 M H₂SO₄. Absorbance was measured with an ELISA reader. Each individual data point was performed in triplicate. There was minimal background binding of the MBL to wells not containing virus.

The experiments demonstrated specific binding of MBL to the SARS-CoV.

Assay of SARS-CoV Infectivity

The fetal rhesus kidney cell line (FRhK-4) monolayers were prepared in 96-well plates and grown to confluence. These layers were then infected with SARS-CoV (100 TCID50) previously treated with MBL (0 to 10 μg/mL) for 2 hours at 37° C. After 2-3 days the monolayers were examined for cythophatic effects (CPE) under light microscope.

The experiments demonstrated a MBL concentration dependent inhibition of infectivity to the SARS-CoV.

### REFERENCES

1. Weis W I, Taylor M E and Drickamer K (1998) The C-type lectin superfamily in the immune system. Immunological Reviews 163: 19-34
2. Holmskov, U., Malhotra, R., Sim, R. B., and Jensenius, J. C. (1994) Collectins: collagenous C-type lectins of the innate immune defense system. Immunol. Today 15:67-74.
3. Turner, M. W. (1996) Mannose-binding lectin: the pluripotent molecule of the innate immune system. Immunol. Today 17:532-540.
4 Janeway C A, Travers P, Walport M and Capra J D (1999) Immunobiology, the immune system in health and disease, Fourth Edition, Churchill Livingstone)
5. Matsushita, M. and Fujita, T (1992). Activation of the classical complement pathway by mannose-binding protein in association with a novel C1s-like serine protease. J. Exp. Med. 176:1497-1502.
6. Thiel S, Vorup-Jensen T, Stover C M, Schwaeble W, Laursen S B, Poulsen K, Willis A C, Eggleton P, Hansen S, Holmskov U, Reid K B and Jensenius J C (1997) A second serine protease associated with mannan-binding lectin that activates complement. Nature, 386(6624): 506-510
7. Stover C M, Thiel S, Thelen M, Lynch N J, Vorup-Jensen T, Jensenius J C and Schwaeble W J (1999) Two constituents of the initiation complex of the mannan-binding lectin activation pathway of complement are encoded by a single structure gene. J Immunol 162: 3481-3490
8. Thiel S, Holmskov U, Hviid L, Laursen S B and Jensenius J C (1992) The concentration of the C-type lectin, mannan-binding protein, in human plasma increases during an acute phase response. Clin Exp Immunol 90: 31-35
9. Madsen, H. O., Garred, P., Kurtzhals, J. A., Lamm, L. U., Ryder, L. P., Thiel, S., and Svejgaard, A. (1994) A new frequent allele is the missing link in the structural polymorphism of the human mannan-binding protein. Immunogenetics 40:3744.
10. Summerfield J A, Ryder S, Sumiya M, Thursz M, Gorchein A, Monteil M A and Turner M W (1995) Mannose binding protein gene mutations associated with unusual and severe infections in adults. Lancet 345: 886-889
11. Garred P, Madsen H O, Hofmann B and Svejgaard A (1995) Increased frequency of homozygosity of abnormal mannan binding protein alleles in patients with suspected immunodeficiency. Lancet 346: 941-943
12. Summerfield J A, Sumiya M, Levin M and Turner M W (1997) Association of mutations in mannose-binding protein gene with childhood infection in consecutive hospital series. BioMed J 314: 1229-1232
13. van Emmerik, L C, Kuijper, E J, Fijen, CAP, Dankert, J, and Thiel, S (1994) Binding of mannan-binding protein to various bacterial pathogens of meningitis. Clin. Exp. Immunol. 97:411-416.
14. Jack D L, Dodds A W, Anwar N, Ison C A, Law A, Frosch M, Turner M W and Klein N J (1998) Activation of complement by Mannose-binding lectin on isogenic mutants of Neisseria meningitidis serogroup B. J Immunol 160:1346-1353
15. Miller, M. E., Seals, J., Kaye, R., and Levitsky, L. C. (1968) A familial, plasma-associated defect of phagocytosis. A new case of recurrent bacterial infections. The. Lancet :60-63.
16. Super, M., Thiel, S., Lu, J., Levinsky, R. J., and Turner, M. W. (1989) Association of low levels of mannan-binding protein with a common defect of opsonisation. Lancet 2:1236-1239.
17. Nielsen, S. L., Andersen, P. L., Koch, C., Jensenius, J. C., and Thiel, S. (1995) The level of the serum opsonin, mannan-binding protein in HIV-1 antibody-positive patients. Clin. Exp. Immunol. 100:219-222.
18. Christiansen, O. B., Kilpatrick, D. C., Souter, V., Varming, K., Thiel, S., Jensenius, J. C. (1999) Mannan-binding lectin deficiency is associated with unexplained re-current miscarriage. Scand. J. Immunol., 49, 193-196
19. Garred, P, Harboe, M, Oettinger, T, Koch, C, and Svejgaard, A (1994) Dual role of mannanbinding protein in infections: Another case of heterosis ? Eur. J. Immunogen. 21:125-131.
20. Hoal-Van Helden E G, Epstein J, Victor T C, Hon D, Lewis L A, Beyers N, Zurakowski D, Ezekowitz A B, Van Helden P D (1999) Mannose-binding protein B allele confers protection against tuberculous meningitis. Pediatr Res 45:459-64
21. Fischer, P B, Ellerman-Eriksen, S, Thiel, S, Jensenius, J C, and Mogensen, S C (1994) Mannan-binding protein and conglutinin mediate enhancement of herpes simplex virus type-2 infection in mice. Scand J Immunol 39:439445.
22. Valdimarsson H, Stefansson M, Vikingsdottir T, Arason G J, Koch C, Thiel S and Jensenius J C (1998) Reconstitution of opsonizing activity by infusion of mannan-binding lectin (MBL) to
24. Pizzo, P A (1993), Management of fever in patients with cancer and treatment-induced neutropenia, N Eng J Med, 328, 1323-1332.
25. Aittoniemi, J., Miettinen, A., Laine, S., Sinisalo, M., Laippala, P., Vilpo, L, Vilpo, J. (1999), Opsonising immunoglobulins and mannan-binding lectin in chronic lymphocytic leukemia, Leuk Lymphoma Jul;34(34):3815
26. Lehmbecher T, Venzon D, de Haas M, Chanock S J, Kuhl J. (1999) Assessment of measuring circulating levels of interleukin6, interleukin8, Creactive protein, soluble Fc gamma receptor type III, and mannosebinding protein in febrile children with cancer and neutropenia. Clin Infect Dis, Aug;29(2):4149.
27. Lu, J., Thiel, S., Wiedemann, H., Timpl, R. & K. B. M. Reid (1990) Binding of the pentamer/hexamer forms of mannan-binding protein to zymosan activates the proenzyme C1r₂C1s₂ complex, of the classical pathway of complement without involvement of C1q. J. Immunol. 144:2287-2294.
28. Sastry, K., Herman, G. A., Day, L., Deignan, E., Bruns, G., Morton, C. C. & R. A. B. Ezekowitz (1989) The human mannose-binding protein gene. J. Exp. Med. 170:1175-1189.
29. Lipscombe, R. J., Sumiya, M., Summerfield, J. A. & M. W. Turner (1995) Distinct physicochemical characteristics of human mannose-binding protein expressed by individuals of differing genotype. Immunology 85:660-667.

1-31. (canceled)

32. A method of using an MBL composition for preventing and/or reducing SARS in an individual, the method comprising the steps of:
a) determining serum levels of MBL in an individual,
b) estimating the probability of the occurrence of a significant clinical SARS in the individual, and optionally,
c) administering an MBL composition to the individual.

33-34. (canceled)

35. A method for preventing or treating Severe Acute Respiratory Syndrome, comprising administering an effective amount of a composition comprising at least one collectin and/or ficolin subunit, or at least one collectin and/or ficolin oligomer comprising the collectin and/or ficolin subunit, to an individual in need thereof.

36. The method of claim 35, wherein the composition comprises at least one mannan-binding lectin (MBL) oligomer comprising the at least one mannan-binding lectin (MBL) subunit.

37. The method of claim 36, wherein said oligomer is preferably selected from the group of oligomers consisting of tetramers, pentamers and/or hexamers.

38. The method of claim 35, wherein the individual has a serum level of MBL in excess of 10 ng/ml serum.

39. The method of claim 35, wherein the individual has a serum level of MBL in excess of 50 ng/ml serum.

40. The method of claim 38, wherein the serum MBL level is the functional serum MBL level.

41. The method of claim 39, wherein the serum MBL level is the functional serum MBL level.

42. The method of claim 35, further comprising administering an antimicrobial medicament capable of attenuation and/or elimination a microbial species.

43. The method of claim 42, further comprising administering an antibacterial medicament capable of bacterial attenuation and/or elimination.

44. The method of claim 35, wherein the MBL subunit or the MBL oligomer is produced in a native host organism.

45. The method of claim 44, wherein the native host organism is a human cell natively expressing the MBL subunit or the MBL oligomer.

46. The method of claim 35, wherein the MBL subunit or MBL oligomer is produced by a host organism not natively expressing an MBL polypeptide.

47. The method of claim 35, wherein the MBL subunit or the MBL oligomer is produced by a method comprising at least one step of recombinant DNA technology in vitro.

48. The method of claim 46, wherein the production of the MBL subunit or the MBL oligomer is controlled by an expression control sequence not natively associated with MBL polypeptide expression.

49. The method of claim 47, wherein the production of the MBL subunit or the MBL oligomer is controlled by an expression control sequence not natively associated with MBL polypeptide expression.

50. The method of claim 44, wherein the MBL subunit or the MBL oligomer is isolated from the host organism.

51. The method of claim 50, wherein the MBL subunit or the MBL oligomer is isolated by a method comprising at least one step involving affinity chromatography.

52. The method of claim 50, wherein the affinity chromatography step is capable of isolating MBL tetramers, pentamers and/or hexamers from a composition further comprising additional MBL oligomers and/or MBL subunits.

53. The method of claim 35, wherein the MBL subunit and/or the MBL oligomer is free from any impurities naturally associated with the MBL when produced in a native host organism.

54. The method of claim 35, wherein the MBL subunit is a mammalian MBL subunit.

55. The method of claim 54, wherein the mammalian MBL subunit is a human MBL subunit.

56. The method of claim 35, wherein the medicament is administered to the individual prior to another treatment.

57. The method of claim 35, wherein the administration is a booster of MBL serum levels in an individual having MBL serum levels above a predetermined minimum MBL serum level of 10 ng/ml.

58. The method of claim 57, wherein the individual has MBL serum levels below a predetermined maximum MBL serum level of 500 ng/ml.

59. The method of claim 35, wherein the individual has serum levels of MBL in excess of 75 ng/ml.

60. The method of claim 35, wherein the individual has serum levels of MBL in excess of 100 ng/ml.

61. The method of claim 35, wherein the individual has serum levels of MBL in excess of 150 ng/ml.

62. The method of claim 35, wherein the individual has serum levels of MBL below 500 ng/ml.

63. The method of claim 35, wherein the individual has serum levels of MBL below 400 ng/ml.

64. The method of claim 35, wherein the individual has serum levels of MBL below 300 ng/ml.

65. The method of claim 35, wherein serum or plasma levels of MBL in the individual are determined by quantitative analysis.

66. The method of claim 65, wherein the analysis comprises at least one of ELISA, TRIFMA, RIA or nephelometry.
